SEQUENCE LISTING (38 sequences)

SEQ ID NO: 1 - DNA, 528 bp, Homo sapiens
atgccgttaccgccacacccgggtcatccaggttatatcaacttctcttacgaggtcttgacccctttaaaatggtaccaatccatccgtcctccgtatcctagttatggctatgaacctatgggtggatggttgcaccatcagattatccctgttttaagtcagcaacacccgcccactcatacattacagcctcaccatcacattcctgtggtacctgcccaacagccggttattcctcaacagccaatgatgccagtgccgggtcagcattctatgaccccaattcaacatcatcagcctaatttaccgccacctgcacaacagccgtaccaaccccaacccgtacaaccgcagccacatcaaccgatgcagccgcagccgccagtgcatccgatgcagcctctgccacctcaaccgccattaccgccaatgtttcctatgcaaccgcttccaccgatgttgccggatttgacgctggaagcctggccctctacggataaaaccaaacgtgaagaggttgactaa

SEQ ID NO: 2 - DNA, 324 bp, Homo sapiens
gtgccgttctttccgcagcaaagcggtaccccaggcatggcatcgctgagcctggagaccatgcgccagttaggtagtttacagcgcctgaacacgctgtcgcaatatagtcgttatggcttcggcaaatcttttaattccttgtggatgcatggcctgttgccgccacactctagcctgccttggatgcgtcctcgcgaacatgaaacccagcaatacgaatactccttgcctgttcacccaccgccgctgccgtcccagccgagcctgaaaccgcagcaaccgggtctgaaaccgtttttacagagcgcggccgcgacgtaa

SEQ ID NO: 3 - DNA, 855 bp, Artificial Sequence
  Description of Artificial Sequence Synthetic polynucleotide
atgccgttaccgccacacccgggtcatccaggttatatcaacttctcttacgaggtcttgacccctttaaaatggtaccaatccatccgtcctccgtatcctagttatggctatgaacctatgggtggatggttgcaccatcagattatccctgttttaagtcagcaacacccgcccactcatacattacagcctcaccatcacattcctgtggtacctgcccaacagccggttattcctcaacagccaatgatgccagtgccgggtcagcattctatgaccccaattcaacatcatcagcctaatttaccgccacctgcacaacagccgtaccaaccccaacccgtacaaccgcagccacatcaaccgatgcagccgcagccgccagtgcatccgatgcagcctctgccacctcaaccgccattaccgccaatgtttcctatgcaaccgcttccaccgatgttgccggatttgacgctggaagcctggccctctacggataaaaccaaacgtgaagaggttgacggatccgtgccgttctttccgcagcaaagcggtaccccaggcatggcatcgctgagcctggagaccatgcgccagttaggtagtttacagcgcctgaacacgctgtcgcaatatagtcgttatggcttcggcaaatcttttaattccttgtggatgcatggcctgttgccgccacactctagcctgccttggatgcgtcctcgcgaacatgaaacccagcaatacgaatactccttgcctggtcacccaccgccgctgccgtcccagccgagcctgaaaccgcagcaaccgggtctgaaaccgtttttacagagcgcggccgcgacgtaa

SEQ ID NO: 4 - protein, 284 aa, Artificial Sequence
  Description of Artificial Sequence Synthetic polypeptide
MPLPPHPGHPGYINFSYEVLTPLKWYQSIRPPYPSYGYEPMGGWLHHQIIPVLSQQHPPTHTLQPHHHIPVVPAQQPVIPQQPMMPVPGQHSMTPIQHHQPNLPPPAQQPYQPQPVQPQPHQPMQPQPPVHPMQPLPPQPPLPPMFPMQPLPPMLPDLTLEAWPSTDKTKREEVDGSVPFFPQQSGTPGMASLSLETMRQLGSLQRLNTLSQYSRYGFGKSFNSLWMHGLLPPHSSLPWMRPREHETQQYEYSLPVHPPPLPSQPSLKPQQPGLKPFLQSAAAT

SEQ ID NO: 5 - protein, 175 aa, Homo sapiens
MPLPPHPGHPGYINFSYEVLTPLKWYQSIRPPYPSYGYEPMGGWLHHQIIPVLSQQHPPTHTLQPHHHIPVVPAQQPVIPQQPMMPVPGQHSMTPIQHHQPNLPPPAQQPYQPQPVQPQPHQPMQPQPPVHPMQPLPPQPPLPPMFPMQPLPPMLPDLTLEAWPSTDKTKREEVD

SEQ ID NO: 6 - protein, 107 aa, Homo sapiens
VPFFPQQSGTPGMASLSLETMRQLGSLQRLNTLSQYSRYGFGKSFNSLWMHGLLPPHSSLPWMRPREHETQQYEYSLPVHPPPLPSQPSLKPQQPGLKPFLQSAAAT

SEQ ID NO: 7 - DNA, 78 bp, Artificial Sequence
  Description of Artificial Sequence Synthetic primer
atgccgttaccgccacacccgggtcatccaggttatatcaacttctcttacgaggtcttgacccctttaaaatggtac

SEQ ID NO: 8 - DNA, 78 bp, Artificial Sequence
  Description of Artificial Sequence Synthetic primer
aaccatccacccataggttcatagccataactaggatacggaggacggatggattggtaccattttaaaggggtcaag

SEQ ID NO: 9 - DNA, 78 bp, Artificial Sequence
  Description of Artificial Sequence Synthetic primer
gaacctatgggtggatggttgcaccatcagattatccctgttttaagtcagcaacacccgcccactcatacattacag

SEQ ID NO: 10 - DNA, 78 bp, Artificial Sequence
  Description of Artificial Sequence Synthetic primer
ttggctgttgaggaataaccggctgttgggcaggtaccacaggaatgtgatggtgaggctgtaatgtatgagtgggcg

SEQ ID NO: 11 - DNA, 76 bp, Artificial Sequence
  Description of Artificial Sequence Synthetic primer
cggttattcctcaacagccaatgatgccagtgccgggtcagcattctatgaccccaattcaacatcatcagcctaa

SEQ ID NO: 12 - DNA, 78 bp, Artificial Sequence
  Description of Artificial Sequence Synthetic primer
tggctgcggttgtacgggttggggttggtacggctgttgtgcaggtggcggtaaattaggctgatgatgttgaattgg

SEQ ID NO: 13 - DNA, 78 bp, Artificial Sequence
  Description of Artificial Sequence Synthetic primer
aacccgtacaaccgcagccacatcaaccgatgcagccgcagccgccagtgcatccgatgcagcctctgccacctcaac

SEQ ID NO: 14 - DNA, 78 bp, Artificial Sequence
  Description of Artificial Sequence Synthetic primer
gtcaaatccggcaacatcggtggaagcggttgcataggaaacattggcggtaatggcggttgaggtggcagaggctgc

SEQ ID NO: 15 - DNA, 72 bp, Artificial Sequence
  Description of Artificial Sequence Synthetic primer
ccgatgttgccggatttgacgctggaagcctggccctctacggataaaaccaaacgtgaagaggttgactaa

SEQ ID NO: 16 - DNA, 32 bp, Artificial Sequence
  Description of Artificial Sequence Synthetic primer
aaagcgggatccatgccgttaccgccacaccc

SEQ ID NO: 17 - DNA, 35 bp, Artificial Sequence
  Description of Artificial Sequence Synthetic primer
tttgcgaagcttttagtcaacctcttcacgtttgg

SEQ ID NO: 18 - DNA, 33 bp, Artificial Sequence
  Description of Artificial Sequence Synthetic primer
gtgtccagcgcatatgccgttaccgccacaccc

SEQ ID NO: 19 - DNA, 39 bp, Artificial Sequence
  Description of Artificial Sequence Synthetic primer
caacggtgccggatccttagtcaacctcttcacgtttgg

SEQ ID NO: 20 - DNA, 70 bp, Artificial Sequence
  Description of Artificial Sequence Synthetic primer
gtgccgttctttccgcagcaaagcggtaccccaggcatggcatcgctgagcctggagaccatgcgccagt

SEQ ID NO: 21 - DNA, 71 bp, Artificial Sequence
  Description of Artificial Sequence Synthetic primer
agccataacgactatattgcgacagcgtgttcaggcgctgtaaactacctaactggcgcatggtctccagg

SEQ ID NO: 22 - DNA, 72 bp, Artificial Sequence
  Description of Artificial Sequence Synthetic primer
tcgcaatatagtcgttatggcttcggcaaatcttttaattccttgtggatgcatggcctgttgccgccacac

SEQ ID NO: 23 - DNA, 71 bp, Artificial Sequence
  Description of Artificial Sequence Synthetic primer
ttcgtattgctgggtttcatgttcgcgaggacgcatccaaggcaggctagagtgtggcggcaacaggccat

SEQ ID NO: 24 - DNA, 72 bp, Artificial Sequence
  Description of Artificial Sequence Synthetic primer
acatgaaacccagcaatacgaatactccttgcctgttcacccaccgccgctgccgtcccagccgagcctgaa

SEQ ID NO: 25 - DNA, 69 bp, Artificial Sequence
  Description of Artificial Sequence Synthetic primer
cgtcgcggccgcgctctgtaaaaacggtttcagacccggttgctgcggtttcaggctcggctgggacgg

SEQ ID NO: 26 - DNA, 32 bp, Artificial Sequence
  Description of Artificial Sequence Synthetic primer
tagccgggatccgtgccgttctttccgcagca

SEQ ID NO: 27 - DNA, 35 bp, Artificial Sequence
  Description of Artificial Sequence Synthetic primer
atggcgaagcttttacgtcgcggccgcgctctgta

SEQ ID NO: 28 - DNA, 36 bp, Artificial Sequence
  Description of Artificial Sequence Synthetic primer
gtgtccagcgcatatggtgccgttctttccgcagca

SEQ ID NO: 29 - DNA, 39 bp, Artificial Sequence
  Description of Artificial Sequence Synthetic primer
caacggtgccggatccttacgtcgcggccgcgctctgta

SEQ ID NO: 30 - DNA, 858 bp, Artificial Sequence
  Description of Artificial Sequence Synthetic polynucleotide
atggtgccgttctttccgcagcaaagcggtaccccaggcatggcatcgctgagcctggagaccatgcgccagttaggtagtttacagcgcctgaacacgctgtcgcaatatagtcgttatggcttcggcaaatcttttaattccttgtggatgcatggcctgttgccgccacactctagcctgccttggatgcgtcctcgcgaacatgaaacccagcaatacgaatactccttgcctggtcacccaccgccgctgccgtcccagccgagcctgaaaccgcagcaaccgggtctgaaaccgtttttacagagcgcggccgcgacgggatccatgccgttaccgccacacccgggtcatccaggttatatcaacttctcttacgaggtcttgacccctttaaaatggtaccaatccatccgtcctccgtatcctagttatggctatgaacctatgggtggatggttgcaccatcagattatccctgttttaagtcagcaacacccgcccactcatacattacagcctcaccatcacattcctgtggtacctgcccaacagccggttattcctcaacagccaatgatgccagtgccgggtcagcattctatgaccccaattcaacatcatcagcctaatttaccgccacctgcacaacagccgtaccaaccccaacccgtacaaccgcagccacatcaaccgatgcagccgcagccgccagtgcatccgatgcagcctctgccacctcaaccgccattaccgccaatgtttcctatgcaaccgcttccaccgatgttgccggatttgacgctggaagcctggccctctacggataaaaccaaacgtgaagaggttgactaa

SEQ ID NO: 31 - protein, 285 aa, Artificial Sequence [sequence omitted]

SEQ ID NO: 32 - protein, 12 aa, Artificial Sequence
  Description of Artificial Sequence Synthetic peptide
MRGSHHHHHHGS

SEQ ID NO: 33 - DNA, 15 bp, Artificial Sequence
  Description of Artificial Sequence Synthetic oligonucleotide
catatgccgttaccg

SEQ ID NO: 34 - DNA, 15 bp, Artificial Sequence
  Description of Artificial Sequence Synthetic oligonucleotide
catatggtgccgttc

SEQ ID NO: 35 - DNA, 24 bp, Artificial Sequence
  Description of Artificial Sequence Synthetic oligonucleotide
gccgcgacgggatccatgccgtta

SEQ ID NO: 36 - DNA, 24 bp, Artificial Sequence
  Description of Artificial Sequence Synthetic oligonucleotide
gaggttgacggatccgtgccgttc

SEQ ID NO: 37 - DNA, 18 bp, Artificial Sequence
  Description of Artificial Sequence Synthetic oligonucleotide
ggatccatgccgttaccg

SEQ ID NO: 38 - DNA, 15 bp, Artificial Sequence
  Description of Artificial Sequence Synthetic oligonucleotide
ggatccgtgccgttc

(19) United States
(12) Patent Application Publication
(10) Pub. No.: US 2010/0233783 A1
(43) Pub. Date: Sep. 16, 2010

(54) NOVEL METHOD FOR PROTEIN PURIFICATION

(75) Inventors: Johan Per Henrik Svensson, Lund (SE); Leif Bülow, Lund (SE)

Correspondence Address: RISSMAN HENDRICKS & OLIVERIO, LLP, 100 Cambridge Street, Suite 2101, BOSTON, MA, 02114, US

(73) Assignee: Straumann Holding AG, Basel (CH)

(21) Appl. No.: 11/909,550

(22) PCT Filed: Mar. 23, 2006
(86) PCT No.: PCT/SE06/00359

§ 371 Date: Dec. 21, 2007

Related U.S. Application Data
(60) Provisional application No. 60/664,726, filed on Mar. 24, 2005.

(30) Foreign Application Priority Data
Mar. 24, 2005 (SE) 0500677-0

(51) Int. Cl. C07K 1/14; C07K 14/575; C07K 14/765; C07K 14/62; C07K 14/585; C07K 7/14; C07K 14/665; C07K 14/77; C12N 9/92; C12N 9/26; C07K 14/555; C07K 14/755; C07K 14/78; C12N 9/74; C12N 15/63; C12N 1/21; C07K 14/00

(52) U.S. Cl. 435/201; 530/412; 530/399; 530/364; 530/305; 530/307; 530/316; 530/302; 530/368; 435/234; 530/351; 530/383; 530/356; 530/395; 435/214; 435/320.1; 435/252.33; 530/350

(57) ABSTRACT

Process for purifying a recombinant protein including one or a few procedural steps only. The process combines the step of lysis of the host cell, with the purification of the protein of interest, allowing for a rapid and much more efficient process of purification. The conditions used during the purification process are those of a high temperature and a low pH, allowing for thermostable and acid-resistant recombinant proteins to be isolated from a suspension. The invention also relates to purifying recombinant proteins which are fusion proteins, wherein one part of the protein may be selected from an enamel matrix protein, such as amelogenin.

### FIELD OF THE INVENTION

The present invention relates to a novel process for purifying a recombinant protein, which process is characterised by that it comprises a combined step of cell disruption and primary purification of the protein, allowing for a more rapid and efficient process of protein purification than previously provided by any purification process available within the field of the art.

The recombinant protein is purified employing conditions, which in one step combine a high temperature and a low pH, allowing for a protein which is thermostable and/or resistant to acidic conditions, and/or a protein that is soluble at a low pH and/or at an elevated temperature, or for a fusion protein including at least a fragment of such a protein, to be purified in a manner, which in strong contrast to common state of the art of protein purification today, involves a process with only a few number of steps. The method of the present invention presents a novel procedural step for simultaneously extracting and concentrating a protein of interest from a host cell, at the same time removing practically all, or at least the majority of the host cell proteins.

In particular, the present invention relates to a process for purifying a recombinant protein, wherein host cells are suspended in acetic acid and simultaneously subjected to an incubation at a temperature of between 60-100° C., followed by the protein of interest being collected from the soluble fraction of the solution by any suitable means, such as by centrifugation or filtration.

The present invention thus in one aspect concerns a process for purifying a recombinant protein, wherein said recombinant protein is a thermostable protein and/or an chemostable protein and/or a protein that is soluble at a low pH and/or at an elevated temperature. Such a protein may, in a presently preferred embodiment of the invention, be selected from the group consisting of enamel matrix proteins, e.g. amelin, amelogenin, proline rich non-amelogenins, tuftelin, and tuft proteins.

The present invention furthermore relates to a process for purifying a recombinant protein, wherein said protein is a fusion protein comprising at least a fragment of a recombinant protein which is thermostable and/or resistant to acidic conditions, and/or a protein that is soluble at a low pH and/or at an elevated temperature, which can be selected from the group consisting of enamel matrix proteins, e.g. amelin, amelogenin, proline rich non-amelogenins, tuftelin, and tuft proteins.

Additionally, the invention also encompasses vectors and host cells comprising a recombinant protein suitable for protein purification in a process according to the invention.

### BACKGROUND OF THE INVENTION

Recent advancements in functional and structural genomics have made high-level protein expression mandatory for successful characterisation and production of gene products. The application areas cover among others, production for pharmaceutical purposes (e.g. as drug itself or for high-throughput screens or other assays) and functional analysis, e.g. for structure determinations. A wide variety of expression vectors, including bacterial, yeast, fungal, viral, invertebrate and mammalian culture systems are available. Each of these systems has been used with a varying degree of success to produce proteins for purposes mentioned above. At this end, Escherichia coli and Saccharomyces cerevisiae are among the best studied micro-organisms and have been established as robust and cost-effective hosts for heterologous protein expression of many foreign genes. However, several problems plague high-level expression, among others, stability of mRNA and gene product, post-translational modifications, correct folding, or codon usage, as well as the necessity of a series of costly purification steps. Specifically, purification at industrial levels must supply a protein of sufficient purity in a cost effective manner.

A distinctive problem with the above exemplified hosts for large-scale manufacture of biological products is that they do not excrete products into the medium. Although forced protein excretion can be obtained in certain cases, this might not always be possible, and when it is, proteins are often degraded by the highly aerated and sheared extracellular environment. Effective techniques for cell disruption are therefore required. These include today physical, chemical, enzymatic and mechanical methods. Mechanical methods, such as bead milling, high-pressure homogenization, and microfluidisation are presently employed in the art. However, gentler, specific methods are receiving increasing attention, particularly when used in combination to synergistically exploit the different specificities. Examples for such methods are physical, chemical and enzymatic methods.

Complete disruption of the cell wall and release of the intracellular components requires destruction of the strength-providing components of the wall, namely peptidoglycan in gram-negative or gram-positive bacteria and glucan in yeast. Complete destruction is usually achieved by mechanical means including French press, shaking with glass beads and sonication, bead milling, high-pressure homogenization and microfluidisation (as reviewed by Middelberg A., “Process-scale disruption of microorganisms”, Biotechnology Advances, 1995, Vol. 13, No. 3, pp. 491-551).

Physical methods that can be employed rely on disruption of the wall structure without tearing it away and can e.g. be decompression with rapid gas expansion within the cell, thermolysis, or osmotic shock.

Chemical methods rely on selective interaction of a chemical with components of the wall or membrane and include use of antibiotics, chaotropes, detergents, solvents, hydroxide and/or hypochlorite, and chelating agents.

Enzymatic methods typically involve an enzymatic attack of the peptidoglycan layer in gram-negative bacteria and are mostly conducted in combination with a chemical approach to penetrate the outer membrane of gram-negative bacteria, and/or of the mannoprotein and glucan components of the yeast wall. Typical enzymatic methods employed today are autolysis, phage lysis, and lysis by foreign lytic enzymes.

It is well-known in the art to combine the above-mentioned methods, which often results in a synergistically effective disruption of the host cell. In particular, enzymatic treatment prior to mechanical disruption provides clear benefits. However, the cost implications of such a combinatory treatment and its downstream effects on the quality of the obtained protein must be addressed in each approach separately.

What is more, downstream processing of said released expressed recombinant proteins is another important consideration in the utilisation of cloned gene products. To meet commercial demands, a protein must be economically produced in large quantities and to be useful for therapeutic applications, said protein must often be greater than 99% pure, as determined by a variety of methods including SDS polyacrylamide gel electrophoresis (PAGE) and size exclusion HPLC. In addition, contaminating endotoxin levels, especially derived from gram-negative hosts, must be very low, on the order of less than 1.0 endotoxin units (E:U)/mg protein, as determined by clot gel assay (LAL).

Today, a plethora of different methods are used to purify a recombinant protein in sufficient purity from its host, all characterised by including a multi-step procedure combining a separate cell disruption, followed by at least one purification step and often again followed by several additional purification steps. Unfortunately, many of these methods are inadequate for industrial-scale applications. Problems that frequently occur are high costs, need for use of toxic material, contamination by degradation products and host protein by-products, as well as labour and time intensity. In general, because each step in such a multi-step procedure invariably results in some loss of material, the overall number of steps should be kept to a minimum.

Consequently, several approaches are currently known that reduce the number of steps necessary for an effective purification of recombinantly expressed proteins from their host cells. Some approaches employ a cost effective purification step including heat-treatment of a crudely prepared cell-lysate including the recombinant proteins. Examples of such methods can in particular be found in EP 0289129, WO 2004/048588 and JP 10101696.

Yet another approach reports the utilisation of such heat-treatment of a crude protein extract for inducing the specific denaturation and precipitation of E. coli proteins in a system, wherein small peptides are fused to the C-terminus of a thermostable protein, which protects said peptides from denaturation at 94° C. (see De Marco, A. et al., “Recombinant proteins fused to thermostable partners can be purified by heat incubation.”Journal of Biotechnology 107, (2004) 125-133).

All currently employed processes for purifying recombinant proteins from microorganisms, though; still necessitate at least two distinctly separated procedural steps for the initial disruption of the host cell and for the first crude separation of the heterologous protein from host cell proteins and contaminants.

The aim of the present invention lies in providing a cost-effective and simple method for harvesting a protein of high purity, for the first time effectively combining releasing a heterologous protein from its host cell, separating host cell proteins from the recombinant protein and removing contaminants from the protein solution, in one single procedural step, which may of course be followed by further purification steps, if need for even higher purity of the protein of interest arises. Said method comprises a novel and unexpected combination of cell disruption and purification in one procedural step, thereby considerably reducing the risk for loss of material.

In a particular aspect, one aim of the present invention is to overcome problems well-known in the art, which are encountered trying to purify recombinantly produced enamel matrix protein, such as amelogenins, amelin, enamelin, tuft protein, proteases, and/or albumin.

Enamel matrix proteins and enamel matrix derivatives (EMD), in the form of a purified acid extract of proteins from pig enamel matrix, have previously been described in the patent literature to be able to induce hard tissue formation (i.e. enamel formation, U.S. Pat. No. 4,672,032 (Slavkin)), endorse binding between hard tissues (EP-B-0 337 967 and EP-B-0 263 086), promote open wound healing, such as of skin and mucosa, have a beneficial effect on treatment of infections and inflammatory diseases (EPO 1, 1059934 and EPO II, 01201915.4), induce regeneration of dentin (WO 01/97834), promote the take of a graft (WO 00/53197), induce apoptosis in the treatment of neoplasms (WO 00/53196), and facilitate filling a wound cavity and/or tissue defect following from a procedure and/or trauma, such as a cytoreductive surgery (WO 02/080994), and to restore functional periodontal ligament, cementum and alveolar bone in patients with severe tooth attachment loss (Hammarström et al., 1997, Journal of Clinical Periodontology 24, 658-668).

Furthermore, in studies on cultured periodontal ligament cells (PDL), it was shown that the attachment rate, growth and metabolism of these cells were significantly increased when EMD was present in the cultures. Also, cells exposed to EMD showed increased intracellular cAMP signalling and autocrine production of growth factors, when compared to controls. Epithelial cells on the other hand, although increasing cAMP signalling and growth factor secretion when EMD was present, were inhibited in both proliferation and growth (Lyngstadaas et al., 2001, Journal of Clinical Periodontology 28, 181-188).

Amelogenins, a major constituent of the enamel matrix, are a family of hydrophobic proteins derivable from a single gene by alternative splicing and controlled post secretory processing. They are highly conserved throughout vertebrate evolution and demonstrate a high overall level of sequence homology among all higher vertebrates examined (80%). During cementogenesis in the developing tooth, amelogenin degrades into smaller pieces, and these pieces seem to interact differentially with the surrounding tissue and promote serial steps in the development of the periodontal system. As already described in Fincham et al, (1993), enamel contains a complex of amelogenin proteins, which include components ranging in size from 5-25 kDa. To date, two classes of bovine amelogenin proteins have been described in the size of between 5-6 kDa, namely leucine-rich amelogenin polypeptide (LRAP) and tyrosine-rich amelogenin polypeptide (TRAP). LRAP is translated from a shorter mRNA that has the coding regions from exons 4, 5 and part of 6 deleted during splicing.

Consequently, as an alternative to the rather blunt use of a complete cocktail of enamel matrix proteins, the recombinant production of a specifically selected active enamel protein, such as amelogenin, or a fragment thereof, would be desirable. The obvious benefits of using a highly pure recombinant protein, instead of the isolated porcine EMD, obviously being lack of unwanted side effects and contamination risk, and allowance of a more refined use, e.g. to induce specific steps during periodontal development, such as de novo bone formation or cementogenesis, and/or to mimic them in medical treatments.

Meeting the above specific demands, the present invention for the first time discloses a method for expressing and purifying a recombinantly produced enamel matrix protein, furthermore disclosing the surprising potential for employing said recombinant enamel matrix protein for expressing and purifying other recombinant proteins and/or peptides of interest with a method as described herein.

The present invention consequently also relates to a purified recombinant enamel matrix protein, which can be harvested employing a method according to the present invention and to the use of such a recombinant enamel matrix protein as a medicament.

### DISCLOSURE OF THE INVENTION

### Protein Purification Process

In the field of the art, production of a pure recombinant protein is a multi-step process including cell cultivation and harvesting, cell disruption, and various steps of protein separation and purification. The purification process generally starts with a crude extract containing the protein of interest (POI) together with a lot of contaminants, mainly host cell proteins. The crude extract is obtained by disruption of the host cells using mechanical, physical, chemical or enzymatic methods, followed by separation and removal of the insoluble fraction. The following separation of the POI from the host cell proteins in the crude extract is a multi step purification process that starts with a couple of primary steps, often chromatography based, in order to remove the bulk of contaminating proteins. This is again followed by a number of polishing steps, to further remove unwanted proteins and other contaminants, in order to obtain the POI with sufficient purity.

Initially the purification of amelogenin from E. coli cells was therefore approached by disrupting the cells by sonication in different solutions, in order to create a crude extract. It was found that sonication in 3% HAc precipitated a lot of the host cell proteins (FIG. 2). The amelogenin however, was still left in solution and could easily be isolated by removing the insoluble fraction. Sonication in Na-phosphate buffer (Buffer A) left practically all proteins soluble, suggesting that this buffer is a poorer choice for amelogenin purification. Subsequent heat treatment at 80° C. of the crude extracts showed that amelogenin dissolved in 3% HAc was resistant to the high temperature, but when Na-phosphate buffer was used, this temperature resistance was not observed and amelogenin precipitated together with many of the host cell proteins. This surprisingly indicated that the temperature resistance of amelogenin is pH dependent.

In order to establish, if the isolation of recombinant amelogenin could be made even easier, i.e. employing fewer procedural steps, untreated cells were heated to 80° C. in 3% HAc, without prior disruption. The aim was to disrupt the cells by the heat treatment, and let the host cell proteins precipitate by the low pH and the heat, leaving only amelogenin left in solution. This procedure would for the first time combine cell disruption with purification of recombinant amelogenin. FIG. 3 shows the results using this approach compared to using a separate step for cell disruption, and demonstrates that the combined cell disruption/purification step surprisingly generates a more pure amelogenin. A separate cell disruption step, followed by heat treatment of the crude extract was thus not only shown to be more laborious than direct heat treatment of the harvested cells, but also to release less pure amelogenin. What is more, the purity of the crude extract obtained from sonication of the cells in 3% HAc was shown not to be significantly improved by the subsequent heat treatment, suggesting that it is potentially rather the low pH more than the high temperature that denatures the host cell proteins. Nevertheless, a heating treatment is still desirable in a purification process, since it helps to degrade other contaminants and provides sterilization of the protein of interest.

The novel purification process described herein, comprising to heat treat cells in an acidic solution can obviously also be used for other recombinantly expressed proteins which are thermostable and/or resistant to acidic conditions, and/or proteins that are soluble at a low pH and/or at an elevated temperature. The protein of interest should be soluble, but need not necessarily to be active, in the solution used and/or at the high temperature. As will be anticipated by the person skilled in the art, the exact temperature and pH of the solution used will of course have to be modified for the specific protein of interest. This method effectively generates a POI almost free from host cell proteins in only one step, starting with untreated cells, and will be very simple to perform also in a large scale. The total purification process will be significantly shortened, since cell disruption and primary purification are comprised effectively in one procedural step.

Relating to yet another embodiment of the present invention, purification of an amelogenin-amelin fusion protein was carried out in same manner as for amelogenin by heat treatment of the cells at 80° C. in 3% HAc. This procedure was also demonstrated to be successful regarding the purification of the fusion protein (FIG. 5), and again, a very pure protein was obtained from the host cells by only one step. It is thus envisioned that purification of other proteins and/or peptides of interest, using heat treatment at low pH, are to be promoted by fusing the POI to amelogenin, or at least a fragment of an amelogenin, even if the POI alone could not withstand the treatment according to the present invention. Thus, amelogenin constitutes an attractive fusion partner, both to facilitate expression and purification of a wide variety of proteins of interest. In this context, it should of course again be obvious to the person skilled in the art, that amelogenin stands representative for any protein which is equally thermostable and/or resistant to acidic conditions, and/or a protein that is soluble at a low pH and/or at an elevated temperature.

Typically, a protein and/or peptide of interest (POI) to be expressed and/or purified using a method according to the above can be selected from the group consisting of small proteins, enamel matrix proteins, hormones, parathyroid hormones, growth hormones, gonadotropins, insulin, ACTH, prolactin, placental lactogen, melanocyte stimulating hormone, thyrotropin, calcitonin, enkephalin, angiotensin, cytokines human serum albumin, bovine serum albumin, ovalbumin, glucose isomerase, α-amylase, and endo-β-glucanase, growth hormone (GH), IGF-1, IGF-2, PTH, PGE₂, TGF-beta, TGF-alpha, bEGF, EGF, PDGF-AB, PDGF-BB, osteoprotegerin (OPG), osteopontin (OP), FGF-1, FGF-2, thyroid hormone, BMP-2, BMP-3, BMP-4, BMP-6, BMP-7, VEGF, 1.25(OH)₂ vitamin D₃, Caclitonin, IFN-gamma, OCN (osteocalcin), ON (osteonectin), OP-1 (osteogenic protein-1), NGF, collagen, fibronectin, fibrinogen, thrombin, factor XIII.

Purification of recombinant human amelogenin, or any protein which is equally thermostable and/or resistant to acidic conditions, and/or a protein that is soluble at a low pH and/or at an elevated temperature, and amelogenin-amelin fusion protein, or indeed a plethora of proteins of interest fused to amelogenin, or to any protein which is equally thermostable and/or resistant to acidic conditions, and/or a protein that is soluble at a low pH and/or at an elevated temperature, can thus be carried out by a simple method comprising relatively few procedural steps, wherein the protein expressing cells (i.e. the host cells) are subjected to a heat treatment in a low pH solution. This combined process in itself generates proteins practically free from contaminating host cell proteins. The simplicity, ease and speed of this method should make it an attractive alternative in production of many recombinant proteins. Furthermore, it is not necessary to remove cell debris before the primary purification, and the whole process may be carried out in one vessel.

Consequently, one aspect of the present invention lies in providing a cost-effective and simple process for harvesting a broad variety of recombinantly produced proteins of high purity, including releasing a heterologous protein from a host cell, separating host cell proteins from the heterologous protein and removing contaminants from the protein solution. Such a method is characterised by comprising a novel combined cell disruption and purification step as described herein.

Accordingly, the invention relates to a process for purifying a recombinant protein, which process comprises suspending host cells expressing said protein in acetic acid and incubating said suspension at a temperature of between 60-100° C., separating, and collecting the soluble fraction of the suspension, containing the recombinant protein.

In the present context, the term “process” may be used interchangeably with the terms “method” or “procedure” and refers in particular to any assembly and/or order of procedural steps for purifying a recombinantly produced protein as disclosed by the present invention.

The term “pure” or “purify” relates to the level of purity of the protein, or the level of purity to be achieved by a process of purification according to the invention, which contains only minor amounts of other proteins, which normally contaminate a whole cell lysate.

The term a “multi-step” process is in the present context employed to describe a process for purifying a protein and/or a peptide, which comprises a series of costly and/or laborious and/or time-consuming and/or technically complicated purification steps. As a general rule, a multi-step process, in contrast to the in the present invention described process with one or only a few number of steps, comprises at least two distinctly separated procedural steps for the initial disruption of the host cell and for the first crude separation of the heterologous protein from host cell proteins and contaminants. Typically, these steps are again followed by at least one purification step. Although both the multi-step process and the process related to in the present invention may further comprise several additional purification steps, if need for even higher purity of the protein of interest arises, this is not essential for the process described in the present invention. The process of the present invention, generating substantially pure recombinant proteins which are practically free from contaminating host cell proteins, can actually be performed in essentially a single procedural step.

Pure proteins in the present context are understood to may be mixed with carriers or diluents, which will not interfere with the intended purpose of the peptide, which still is to be regarded as substantially isolated.

A substantially “pure” protein according to the invention generally comprises a protein which is at least 90% pure, such as at least 90, 91, 92, 93, 94, 95, 96, 97, 98, 99 or 100% pure. Such purity may be established by methods known in the art, such as, but not limited to, SDS polyacrylamide gel electrophoresis (PAGE) and size exclusion HPLC.

A “recombinant” protein, is a protein which has been produced by an in vivo recombinant technology which may comprise a cell-based expression system comprising a host cell, such as an eukaryotic and/or a prokaryotic cell, and an expression system (i.e. a vector), such as a phage or a plasmid, which plasmid comprises an origin or replication, a gene of interest to be amplified, and optionally a selectable marker to allow for selection of transformants (i.e. host cells which have obtained the plasmid).

A protein which is purifiable employing a process according to the present invention is typically termed to be a recombinant protein. This term is intended to be used interchangeably with “target protein”, “protein of interest”, “POI”, “heterologue protein” or “heterologous protein”.

Proteins are biological macromolecules constituted by amino acid residues linked together by peptide bonds. Typically, proteins have 50-800 amino acid residues and hence have molecular weights in the range of from about 6,000 to about several hundred thousand Dalton or more. Small proteins are called peptides, oligopeptides or polypeptides. Such proteins may range in size from about 1 to about 100 amino acids. It should be emphasized, that in the present context, the term “protein” may refer to proteins of any size and length, such as peptides, oligopeptides, polypeptides, as well as fragments and derivatives thereof.

Amino acid sequence is in the present context the precisely defined linear order of amino acids (including both coded and/or non-coded amino acids) in a peptide fragment, peptide, protein or polypeptide. An amino acid is any organic compound containing an amino (—NH₂) and a carboxyl (—COOH) group. Amino acids can be in either L- or D-form. There are at present 22 known coded α-amino acids from which proteins are synthesized during ribosomal translation of mRNA. Additionally, a vast number of non-coded amino acids are constantly emerging. Both coded and non-coded amino acids can of course be part of the amino acid sequences, peptide fragments, peptides, proteins and/or polypeptides included in the present invention.

The term “non-peptide analogue” is in the present context employed to describe any amino acid sequence comprising at least one non-coded amino acid and/or having a backbone modification resulting in an amino acid sequence without a peptide linkage, i.e. a CO—NH bond formed between the carboxyl group of one amino acid and the amino group of another amino acid.

Furthermore, the present amino acid sequences may either be amidated or occur as free acids. A “cell-based expression system”, according to the invention, refers to a system which comprises host cell(s), a suspension media suitable for the purpose of use, and an expression system (i.e. a vector) which is to be introduced/is introduced in the host cell. The expression system may comprise a plasmid, phage or any other suitable expression system, which carries the gene encoding the protein of interest.

In the context of the present invention, a “cell culture” refers to a culture of cells, such as any of the host cells according to the present invention, which are present in an artificial (e.g. in vitro, in situ) environment, such as a media suspension, or on a solid or semi-solid medium. A “cell culture” may refer to a culture of host cells. “Cell culture” may also be used interchangeably with terms such as “culture system”.

The term “host cell(s)” refers to cell(s) which may be used in a process for purifying a recombinant protein in accordance with the present invention. Such host cells carries the protein of interest (POI). A host cell may also be referred to as a protein expressing cell. A host cell, according to the present invention, may be, but is not limited to, prokaryotic cells, eukaryotic cells, archeobacteria, bacterial cells, insect cells, yeast, mammal cells, and/or plant cells. Bacteria envisioned as host cells can be either gram-negative or gram-positive, e.g. Escherichia coli, Erwinia sp., Klebsellia sp., Lactobacillus sp. or Bacillus subtilis. Typical yeast host cells are selected from the group consisting of Saccharomyces cerevisiae, and Pichia pastoris. In the present context, it should be understood that host cells as starting material for a process of purification according to the present invention do not need to have been subjected to a specific pretreatment to facilitate disruption and/or lysis of the host cell at a later stage. In this context, this is to stand equivalent with the fact that a variety of pretreaments of host cells commonly employed in the art, may be used in a process according to the present invention.

The recombinant cells are cultivated under any physiologically compatible conditions of pH and temperature, in any suitable nutrient medium containing assimilable sources of carbon, nitrogen, and essential minerals that support cell growth. Recombinant protein-producing cultivation conditions will vary according to the type of vector or phage or other means used to introduce the recombinant protein into the host cell. For example, certain expression vectors comprise regulatory regions, which require cell growth at certain temperatures, or addition of certain chemicals to the cell growth medium, to initiate the gene expression which results in the production of the recombinant protein.

In one embodiment of the present invention, a cell culture comprising said host cells, expressing said protein, may be grown in a complex medium. Examples of “complex” media are LB and TB.

In another equally preferred embodiment of the invention, a cell culture comprising said host cells, expressing said protein, may be grown in a defined medium. An example of a “defined” medium is NYAT, M9, M65. Defined media can be supplemented with necessary growth additives such as vitamins and trace metals.

Furthermore, the expression of said recombinant protein in said host cells may also be induced by the addition of lactose to the medium. Additionally, the expression of said recombinant protein in said host cells may be induced by the addition of IPTG (i.e. isopropyl β-D-thiogalactoside) to the medium. IPTG is commonly used to trigger gene expression that is under the control of a lac operator and is particularly used in expression systems for producing protein.

Additionally, antibiotics of any kind, such as tetracycline, ampicillin, kanamycin may be added to maintain the expression vector of interest in a host cell according to the invention.

In another embodiment of the invention, said host cells may be transformed and/or transfected. In yet another embodiment of the invention, said recombinant protein may be expressed transiently and/or stably by the host cells.

According to one embodiment of the invention, said host cells are suspended and/or resuspended in acetic acid, also in the present context referred to as HAc. Said acetic acid may be present in a concentration of about 0.15-1.5%, 0.15-3% or 0.15-5%, such as in a concentration of at least about 0.15; 0.5; 1.0; 1.5; 2.0; 2.5; 3; 3.5; 4; 4, 5 or 5% in said suspension.

A suspension in which said host cells are suspended and/or resuspended in, according to the invention, may display a pH which is below or equal to 5, such as below or equal to 1, 2, 3, 4 or 5. Alternatively, a suspension according to the invention may display a pH below or equal to 3, such as about 0.5; 1.5; 1.75; 2.5; 2.75 or 3. Typically, the range of pH employed in said suspension is from 0.5 to 5, such as from 1 to 4.5; 1.5 to 3.5; 3 to 5, or 1 to 3.

The presently preferred acid for the process for purifying a recombination protein according to the invention is acetic acid. This is not to be understood to be limiting the method of protein purification to the specific use of acetic acid per se. As should be obvious to the person skilled in the art, the acid preferred to achieve a specific pH of the solution in which said host cells are suspended and/or resuspended in will differ for different experimental set-ups and should be construed to comprise any sufficient acid.

Furthermore, a suspension for use in a process for purifying a recombination protein according to the invention will be incubated employing conditions with elevated temperatures, to allow for the initial purification to occur. An incubation period suitable for a process according to the invention may be a period of about 5-40 minutes, such as about 5 to 10, 10 to 20, 10 to 30, 5 to 35 or about 30 to 40 minutes, such as about 5, 10, 15, 18, 20, 25, 30, 35 or 40 minutes. Such an incubation period may also be longer, such as about 30 to 60 minutes. It will be clear to the skilled artisan, that said incubation time is chosen for achieving optimal results, and therefore may vary due to the other conditions (such as temperature variations) used during the process of purification. In one preferred embodiment of the invention, said incubation is performed for at least 5-35 minutes. In another, equally preferred embodiment of the invention, the incubation is performed for at least 20 minutes.

A suspension comprising host cells according to the invention, may be incubated at a temperature of about 60 to 100° C., such as about 60 to 70° C., 65 to 75° C., 70 to 80° C., 75 to 85° C., 80 to 90° C., 85 to 95° C., 90-95° C. or 90 to 100° C., such as selected from about 65, 75, 77, 79, 80, 81, 82, 83, 84, and/or 85° C. In one preferred embodiment of the invention, said incubation is performed at 75 to 85° C. In another, equally preferred embodiment of the invention, the incubation is performed at approximately 80° C. As will be obvious to the skilled person, the temperature might vary slightly due to the experimental set-up. The effect is achieved by the elevation itself.

The term “cell disruption” is in the present context employed to describe the breaking apart of the cell wall and plasma membrane of the host cell to effect the release of intracellular products, thus allowing subsequent recovery. In general, the term is to be understood to include lysis, which is used in the field to describe the rupture of a cell by disrupting its plasma membrane, resulting in the loss of cell contents. The term can be used to relate to procedures resulting in either substantial and/or insubstantial amounts of cytoplasmic material being released. Occurrence of cell disruption can e.g. be shown by viable cell counts that measure bacterial action. With regards to cell disruption of gram-negative organism, this of course also includes the rupture of the outer membrane.

“Separation” of and/or “separating” the protein of interest from cell debris pertaining from the host cells, during a process of the present invention, may be performed by any suitable means, such as by centrifugation or filtration, and/or by using any other standard procedures such as, but not limited to, absorption of the protein of interest to immobilized immunoglobulin, as described by Sjöquist, U.S. Pat. No. 3,850,798 (1974), ion exchange, affinity or gel chromatography, precipitation (e.g. with ammonium sulphate), dialysis, filtration and/or by a combination of these methods.

“Collecting” the soluble fraction during a process of the present invention, may be performed by any suitable means, such as by using a pipette of a suitable size, to obtain the supernatant comprising the protein of interest from the vessel used in the process, or simply by decanting.

A “soluble fraction” according to the invention, refers to a fraction comprising the protein of interest, which protein is made soluble by the specific conditions employed during the process, such as the temperature and the pH chosen, to allow for direct separation of the protein of interest from the cell debris originating from the host cells in the vessel.

In a much preferred embodiment, the invention relates to a process for purifying a recombinant protein, comprising harvesting a cell culture of host cells expressing said protein, resuspending said host cells in acetic acid and incubating said suspension at 60-100° C., separating, and collecting the soluble fraction of the suspension, containing the recombinant protein.

The term “harvesting” or to “harvest” (host) cells of interest in the present context, refers to a procedure of obtaining cells from a cell culture, which cells previously has been allowed to grow in or on a media, which media may have been provided with nutrients and/or other components to facilitate proliferation of the cells. Cells from the cell culture may be harvested by removing them from the media by any means of separation, such as by centrifugation, or by shaking and/or scraping, or by using a specific column, preferably wash the cells, and alternatively resuspending the cells in another solution suitable for the next procedural step.

Consequently, the present invention also concerns a process for purifying a recombinant protein, comprising harvesting and washing a cell culture of host cells expressing said protein, resuspending said host cells in acetic acid and incubating said suspension at 60-100° C., separating, and collecting the soluble fraction of the suspension, containing the recombinant protein.

“Washing” of and/or to “wash” cells for use according to the invention, refers to a procedural step wherein the cells are washed off from any media and/or contaminant or other matter simply by resuspending the cells in any suitable washing media, such as, but not limited to, PBS (i.e. phosphate-buffered saline), or distilled water, and thereafter recover the washed cells from the suspension e.g. by centrifugation and/or filtration.

In another embodiment, said invention relates to a process for purifying a recombinant protein, comprising harvesting and washing a cell culture of host cells expressing said protein, resuspending said host cells in acetic acid and incubating said suspension at 60-100° C., centrifuging said suspension, and collecting the soluble fraction of the suspension, containing the recombinant protein.

In yet another, equally preferred embodiment, the present invention relates to a process for purifying a recombinant protein, comprising harvesting and washing a cell culture of host cells expressing said protein, resuspending said host cells in acetic acid and incubating said suspension at 60-100° C., filtrating said suspension, and collecting the soluble fraction of the suspension, containing the recombinant protein.

The present invention also encompasses a process for purifying a recombinant protein, wherein the soluble fraction of the suspension, containing the recombinant protein, subsequently may be subjected to one or more additional purification step(s).

### Thermostable and Chemostable Proteins

The present invention in one aspect concerns a process for purifying a recombinant protein, wherein said recombinant protein is a thermostable protein and/or a chemostable protein and/or a protein that is soluble at a low pH and/or at an elevated temperature. The present invention furthermore relates to a process for purifying a recombinant protein, wherein said protein is a fusion protein comprising at least a fragment of a recombinant protein which is thermostable and/or resistant to acidic conditions, and/or a protein that is soluble at a low pH and/or at an elevated temperature.

A typical “thermostable protein” is in the present context a protein which is stable in environments with elevated temperatures. Such a protein has a wide range of applications, as it is more robust than other proteins, and it is also characterized by being active in extreme conditions.

Bacteria capable of growing at or above 80° C.-100° C. are generally known as extreme thermophiles or hyperthermophiles. A number of microorganisms have been isolated from extremely hot environments and certain useful compounds have been identified. Examples of thermostable proteins are e.g. thermostable DNA polymerases, proteases, albumins, α-amylase, endo-β-glucanase and glucose isomerase. Such thermostable proteins may all be used in the context of the present invention.

A “chemostable protein”, also referred to as a protein resistant to acidic conditions, is a protein which is resistant to, and which remains stable when, an external and/or internal chemical challenge, e.g. a chemical compound, and/or a chemical condition is encountered. Such a chemical challenge may comprise conditions with a low pH. Accordingly, in one embodiment of the invention, a “chemostable protein” is an acid stable protein, which is capable to withstand conditions with a low pH. Such a protein need not maintain its activity during such conditions, but is able to retain its activity after returning to its normal environment.

A “chemostable” protein retains its activity and/or accurate structure and/or is able to retain its activity after returning to its normal environment, when encountering compound(s) and/or conditions when those compounds and/or conditions typically inhibit proteins, and disrupt the function of non-chemostable proteins.

Thus, in certain embodiments, chemostable proteins, purified by a process according to the invention, comprise a protein that maintains at least about 30% (preferably at least about 60%, 70%, 80%, 90%, 95%, 99%) of its activity and/or accurate structure and/or is able to retain its activity after returning to its normal environment, as measured by any suitable means, such as enzyme activity, binding to a specific antibody or cell and/or affecting cell metabolism, after contact with a chemical compound and/or a chemical condition, sufficient to collectively reduce the activity and/or interfere with the structure of such a protein by at least about 25% (preferably at least about 30%, even more preferably at least about 40%, 50%, 60%, 70%, 80%, 90%, 95%, 99% or any increment therein) relative to the activity of the chemostable protein in the absence of such a chemical compound and/or condition.

In another embodiment of the invention, the chemostability of a protein may also be indicated by the rate of decline of its activity over time when such a protein has encountered a chemical compound and/or condition influencing its activity. These measurements of decline may be graphed to determine a trend of decline in protein activity in the composition over time.

Proteins that are both thermostable protein and chemostable are also envisioned in the present invention. Previously, Midkine, a heparin-binding growth/differentiation factor was found to be heat and acid stable in conditions with a temperature of 97° C. and pH3. (Muramatsu et al, Biochemical and Biophysical Research Communications, Vol. 216, nr 2, p. 574-581, 1995). The experiments were conducted testing the protein activity during conditions of a low pH and a high temperature separately. Lipps et al (2005), demonstrated a highly acid stable and thermostable endo-β-glucanase from the thermoacidophilic archaeon Sulfolobus solfataricus, (Lipps et al, Biochem. 3. (2005), 385, 581-588). This enzyme was shown to have a pH optimum of approximately pH1.8 and a temperature optimum of approximately 80° C. To induce inactivation of the enzyme, preincubation temperatures higher than 85° C. were required. Furthermore, preincubation at a temperature of 80° C. and pH7, rapidly inactivated the enzyme. Saito et al (2000), characterized another chemostable enzyme from Streptomyces olivaceoviridis. The enzyme has sufficient activity between pH5.5 to pH6.0. (Saito et al, Biosci. Biotechnol. Biochem., 64 (5), 940-947, 2000).

In the context of the present invention, heat incubation of E. coli host cells induces specific denaturation and/or precipitation of the E. coli proteins, but not of the thermostable protein of interest expressed by the transformed and/or transfected host cell. In one aspect of the invention, such a thermostable protein may be a fusion protein, wherein one part of the fusion protein is amelogenin and/or amelin and/or fragments thereof.

The present inventors show that amelogenin, alone or as a part of a fusion protein, is highly thermostable allowing for purification in a process using temperature conditions of approximately 60-100° C. Above this, amelogenin is also chemostable, allowing for purification alone or as a part of a fusion protein, during purification conditions with a low pH. The purification process according to the invention, allows for a highly purified protein and/or fusion protein to be produced when said protein and/or fusion protein is resistant to the low pH and the high temperature encountered in the purification process. Accordingly, an example of such a protein is amelogenin, other examples are hydrolase, transferase, oxidoreductase, methyltransferase and glycosidase. Amelogenin has not previously been known to encompass such a rigid thermostability and acid stability, and this finding leads to the possibility of purifying other proteins which may be linked together with amelogenin to form a fusion protein which is stable during such conditions.

Furthermore, in a presently preferred embodiment, the invention relates to a process for purifying a recombinant protein, wherein the recombinant protein is selected from the group consisting of thermostable and/or acid stable proteins, and proteins that are soluble at a low pH and/or at an elevated temperature. Furthermore, the invention also relates to a process for purifying a recombinant protein wherein the protein is a fusion protein comprising at least one fragment of a protein selected from the group consisting of thermostable proteins, acid stable proteins and proteins that are soluble at a low pH and/or at an elevated temperature.

In an equally preferred embodiment, the invention relates to a process for purifying a recombinant protein wherein the protein is a fusion protein comprising at least one fragment of a thermostable protein. In another, equally preferred embodiment of the invention, a recombinant protein purified by a process according to the present invention, is a chemostable protein, or a fragment thereof, which may be a part of a fusion protein according to the invention.

In one embodiment of the invention, a chemostable protein is a protein which is stable at a low pH, such as, but not limited to, amelogenin, amelin, ovalbumin, bovine serum albumin, insulin, albumins, glucose isomerase.

### Enamel Matrix Proteins

A presently preferred embodiment of the invention relates to a process for purifying a recombinant protein, wherein said recombinant protein is an enamel matrix protein. Furthermore, the present invention also relates to a process for purifying a recombinant protein, wherein said protein is a fusion protein comprising at least a fragment of a recombinant protein which is an enamel matrix protein.

Enamel matrix proteins are proteins that normally are present in enamel matrix, i.e. the precursor for enamel (Ten Cate: Oral Histology, 1994; Robinson: Eur. J. Oral Science, January 1998, 106 Suppl. 1:282-91), or proteins which can be obtained by cleavage of such proteins. In general, such proteins have a molecular weight below 120,000 Dalton and include amelogenins, non-amelogenins, proline-rich non-amelogenins and tuftelins.

Prior to cementum formation, enamel matrix proteins are deposited on the root surface at the apical end of the developing tooth-root. There is evidence that the deposited enamel matrix is the initiating factor for the formation of cementum. Again, the formation of cementum in itself is associated with the development of the periodontal ligament and the alveolar bone. Enamel matrix proteins can therefore promote periodontal regeneration through mimicking the natural attachment development in the tooth (Gestrelius S, Lyngstadaas S P, Hammarstrøm L. Emdogain—periodontal regeneration based on biomimicry. Clin Oral Invest 4:120-125 (2000). Enamel matrix proteins are able to induce not only one but an orchestrated cascade of factors, naturally found in tissues developing adjacent to the enamel matrix. They mimic the natural environment of a developing tissue and thus mimic a natural stimulation for tissue regeneration, cell differentiation and/or maturation.

As used herein, the term “enamel matrix protein” refers to a precursor to enamel. In the present context, the term “enamel matrix protein” is used to also encompass derivatives and/or fragments thereof, as well as mixtures thereof, nondiscriminant of their source. Furthermore, the term enamel matrix protein may also encompass synthetic analogous substances of any enamel matrix protein and/or derivative or fragment thereof, as well as enamel matrix protein related polypeptides or proteins. An enamel matrix protein may also refer to a fusion protein comprising an enamel matrix protein, or a portion thereof. Enamel matrix protein may also in the present context be used interchangeably with the term “enamel matrix substance” or “active enamel proteins”.

In the present context, enamel matrix protein derivatives are derivatives of enamel matrix proteins which include one or several enamel matrix proteins or parts or fragments of such proteins. Enamel matrix protein derivatives also include enamel matrix related polypeptides or proteins. The polypeptides or proteins may be bound to a suitable biodegradable carrier molecule, such as polyamine acids or polysaccharides, or combinations thereof.

Examples of enamel matrix proteins for use according to the invention are amelogenins, proline-rich non-amelogenins, tuftelin, tuft proteins, serum proteins, salivary proteins, ameloblastin, sheathlin, and derivatives thereof, and mixtures thereof. A preparation containing an active enamel substance for use according to the invention may also contain at least two of the aforementioned proteinaceous substances. Moreover, other proteins for use according to the invention are found in the marketed product EMDOGAIN® (BIORA AB, Sweden).

As demonstrated in the example section of the present application, one may use cultivated eukaryotic and/or prokaryotic cells modified by DNA-techniques to generate recombinant enamel matrix proteins. As will be understood by the skilled artisan, the recombinant enamel matrix proteins may alternatively be genetically and/or chemically modified (see, e.g., Sambrook, J. et al.: Molecular Cloning, Cold Spring Harbor Laboratory Press, 1989).

In general, the major proteins of an enamel matrix are known as amelogenins. They constitute about 90% w/w of the enamel matrix proteins. The remaining 10% w/w includes proline-rich non-amelogenins, tuftelin, tuft proteins, serum proteins and at least one salivary protein; however, other proteins may also be present such as, e.g., amelin (ameloblastin, sheathlin) which have been identified in association with enamel matrix. Furthermore, the various proteins may be processed in several different sizes (i.e. different molecular weights). Thus, the dominating proteins in enamel matrix, amelogenins, have been found to exist in several different sizes that together form supramolecular aggregates. They are markedly hydrophobic substances that under physiologically conditions form aggregates. They may carry or be carriers for other proteins or peptides. Amelogenin splice variants and proteolytic cleavage products are the main compounds isolated from EMD.

During cementogenesis in the developing tooth, amelogenin, as described above, due to alternative splicing of the primary transcript and the following proteolytic processing of the secreted proteins, degrades into smaller pieces (fragments and polypeptide fragments), and these pieces are hypothesised to interact differentially with the surrounding tissue and promote serial steps in the development of the periodontal system.

The present invention in one embodiment relates to a recombinant protein, purified by a process according to the invention, such as an enamel matrix protein, or a fragment of an enamel matrix protein, or a fusion protein comprising an enamel matrix protein, or a fusion protein comprising at least a portion derived from an enamel matrix protein, for use as a medicament and/or for the use in the manufacture of a pharmaceutical composition for a variety of different medical indications such as inducing bone growth and/or binding between parts of living mineralised tissue, for bonding of a piece of living mineralised tissue to a bonding site on a piece of other living tissue, for preventing and or treating an infection and/or inflammation, for preventing and/or treating an immune system disorder, for endorsing binding between hard tissues, and promoting open wound healing, such as of skin and mucosa, for promoting treatment of infections and inflammatory diseases, inducing regeneration of dentin, promoting the take of a graft, inducing apoptosis in the treatment of neoplasms, facilitating filling a wound cavity and/or tissue defect following from a procedure and/or trauma, such as a cytoreductive surgery and/or coating of a medical device, for preventing downgrowth of fibroblasts and/or epithelial cells into a healing wound, or an implant site. It is to be understood by the skilled artisan, that such a medicament may be included in a pharmaceutical composition for convenient administration and uptake into a body.

A presently preferred embodiment of the invention thus comprises a recombinant protein purified according to the invention, such as an enamel matrix protein, or a fragment of an enamel matrix protein and/or a fusion protein comprising an enamel matrix protein and/or a fusion protein comprising at least a portion derived from an enamel matrix protein, for use as a medicament. In yet another embodiment, the invention relates to a recombinant protein, which protein is purified by a process according to the invention, such as an enamel matrix protein or a fragment thereof and/or a fusion protein comprising an enamel matrix protein and/or a fusion protein comprising at least a portion derived from an enamel matrix protein.

A presently preferred embodiment of the present invention therefore relates to a pharmaceutical, cosmetic and/or therapeutic formulation and/or composition comprising a recombinant protein purified according to the invention, such as an enamel matrix protein and/or a fusion protein comprising an enamel matrix protein and/or a fusion protein comprising at least a portion derived from an enamel matrix protein, as disclosed by the present invention.

Another, equally preferred embodiment further relates to a pharmaceutical and/or therapeutic formulation and/or composition comprising at least a recombinant polypeptide fragment and/or subfragment of a pre-pro amelogenin.

Suspensions of proteins, polypeptides, peptides and/or subfragments and/or polypeptide fragments of a recombinant protein purified according to the invention, such as an enamel matrix protein and/or a fusion protein comprising an enamel matrix protein and/or a fusion protein comprising at least a portion derived from an enamel matrix protein, may be in the context of the present invention, be in a substantially isolated or purified form. It will be understood that the recombinant proteins, polypeptides, peptides and/or subfragments may be mixed with carriers or diluents or be comprised in a pharmaceutical composition, which will not interfere with the intended purpose of the proteins, polypeptides, peptides and/or subfragments, and which will still be regarded as substantially isolated. Such a substantially purified form will generally comprise the recombinant protein, polypeptide, peptide and/or subfragment in a preparation in which more than 90%, e.g. 95%, 96%, 97%, 98% or 99% of the protein in the preparation is a recombinant protein, polypeptide, peptide and/or subfragment according to the invention.

Furthermore, in the present context, any amino acid sequence being at least 70% identical, such as being at least 72%, 75%, 77%, 80%, 82%, 85%, 87%, 90%, 91%, 92%, 93%, 94%, 95%, 96%, 97%, 98%, or 99% identical to the amino acid sequence of a recombinant enamel matrix protein or a fragment of an enamel matrix protein, and/or a fusion protein comprising an enamel matrix protein and/or a fusion protein comprising at least a portion derived from an enamel matrix protein, is also considered to be inside the scope of the present invention.

By a recombinant protein, polypeptide, peptide and/or subfragment thereof having an amino acid sequence at least, for example 95% identical to a reference amino acid sequence, is intended that the amino acid sequence of e.g. the polypeptide is identical to the reference sequence, except that the amino acid sequence may include up to 5 point mutations per each 100 amino acids of the reference amino acid sequence. In other words, to obtain a polypeptide having an amino acid sequence at least 95% identical to a reference amino acid sequence: up to 5% of the amino acids in the reference sequence may be deleted or substituted with another amino acid, or a number of amino acids up to 5% of the total amino acids in the reference sequence may be inserted into the reference sequence. These mutations of the reference sequence may occur at the amino and/or carboxy terminal positions of the reference amino acid sequence or anywhere between those terminal positions, interspersed either individually among amino acids in the reference sequence or in one or more contiguous groups within the reference sequence.

For the present invention, a local algorithm program is best suited to determine identity. Local algorithm programs, (such as Smith-Waterman) compare a subsequence in one sequence with a subsequence in a second sequence, and find the combination of subsequences and the alignment of those subsequences, which yields the highest overall similarity score. Internal gaps, if allowed, are penalized. Local algorithms work well for comparing two multidomain proteins, which have a single domain or just a binding site in common.

Methods to determine identity and similarity are codified in publicly available programs. Preferred computer program methods to determine identity and similarity between two sequences include, but are not limited to, the GCG program package (Devereux, 3 et al (1994)) BLASTP, BLASTN, and FASTA (Altschul, S. F. et al (1990)). The BLASTX program is publicly available from NCBI and other sources (BLAST Manual, Altschul, S. F. et al, Altschul, S. F. et al (1990)). Each sequence analysis program has a default scoring matrix and default gap penalties. In general, a molecular biologist would be expected to use the default settings established by the software program used.

Additionally, any conservative variant and/or analogue of a recombinant protein sequence related to in the present invention, e.g. of a recombinant enamel matrix protein or a fragment of an enamel matrix protein, and/or a fusion protein comprising an enamel matrix protein and/or a fusion protein comprising at least a portion derived from an enamel matrix protein, is by virtue of its functional relationship to said recombinant protein or fragment thereof considered to be inside the scope of the present invention.

A conservative variant of a sequence is in the present context defined as an amino acid sequence which is conserved at least 70%, such as 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, when comparing variants of the same amino acid sequence between different species. The degree of conservation of a variant can, as is well known in the field, be calculated according to its derivation of PAM (see Dayhoff, Schwartz, and Orcutt (1978) Atlas Protein Seq. Struc. 5:345-352), or based on comparisons of Blocks of sequences derived from the Blocks database as described by Henikoff and Henikoff (1992) Proc Natl Acad Sci USA 89(22):10915-9.

Conservative substitutions may be made, for example according to Table 7 below. Amino acids in the same block in the second column and preferably in the same line in the third column may be substituted for each other:

TABLE 7

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Such replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, La-amino butyric acid*, L-g-amino butyric acid*, L-a-amino isobutyric acid*, L-e-amino caproic acid#, 7-amino heptanoic acid*, L-methionine sulfone#*, L-norleucine*, L-norvaline*, p-nitro-L-phenylalanine*, L-hydroxyproline#, L-thioproline*, methyl derivatives of phenylalanine (Phe) such as 4-methyl-Phe*, pentamethyl-Phe*, L-Phe (4-amino)#, L-Tyr (methyl)*, L-Phe (4-isopropyl)*, L-Tic (1,2,3,4-tetrahydroisoquinoline-3-carboxyl acid)*, L-diaminopropionic add # and L-Phe (4-benzyl)*. The notation * is herein utilised to indicate the hydrophobic nature of the derivative whereas # is utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or b-alanine residues. A further form of variation, involves the presence of one or more amino acid residues in peptoid form, which will be well understood by those skilled in the art. For the avoidance of doubt, “the peptoid form” is used to refer to variant amino acid residues wherein the a-carbon substituent group is on the residue's nitrogen atom rather than the α-carbon. Processes for preparing peptides in the peptoid form are known in the art, see for example, Simon R J et al., PNAS (1992) 89(20), 9367-9371 and Norwell D C, Trends Biotechnol. (1995) 13(4), 132-134.

The proteins of an enamel matrix can be divided into a high molecular weight part and a low molecular weight part, which fraction contains acetic acid extractable proteins generally referred to as amelogenins (cf. EP-B-0 337 967 and EP-B-0 263 086).

The low molecular weight part of an enamel matrix has a suitable activity for inducing binding between hard tissues in periodontal defects. In the present context, however, the active proteins are not restricted to the low molecular weight part of an enamel matrix. At present, preferred proteins include enamel matrix proteins such as amelogenins, tuftelin, etc. with molecular weights (as measured in vitro with SDS-PAGE) below about 60,000 Dalton but proteins having a molecular weight above 60,000 Dalton have also promising properties as candidates for promoting connective tissue growth. As mentioned above, the proteins, polypeptides or peptides for use according to the invention typically have a molecular weight of at the most about 120 kDa such as, e.g., at the most 100 kDa, 90 kDa, 80 kDa, 70 kDa or 60 kDa as determined by SDS PAGE electrophoresis.

A preparation of an active enamel substance for use according to the invention may also contain a mixture of active enamel substances with different molecular weights.

Accordingly, it is contemplated that the active enamel substance for use according to the invention has a molecular weight of up to about 40,000 such as, e.g. a molecular weight of between about 5,000 and about 25,000.

The combination of molecular weight amelogenins may be varied, from a dominating 20 kDa compound to an aggregate of amelogenins with many different molecular weights between 40 and 5 kDa, and to a dominating 5 kDa compound. Other enamel matrix proteins such as tuftelin or proteolytic enzymes normally found in enamel matrix can be added and carried by an amelogenin aggregate.

In general, the enamel matrix, enamel matrix derivatives and enamel matrix proteins are hydrophobic substances, i.e. less soluble in water, especially at increased temperatures. In general, these proteins are soluble at non-physiological pH values and at a low temperature such as about 4-20° C., while they will aggregate and precipitate at body temperature (35-37° C.) and neutral pH.

In an especially preferred embodiment of the present invention, a recombinant enamel matrix protein for use according to the invention is selected from the group consisting of any soluble amelogenin based peptide with an amino acid weight between approximately 0, 5-6 kDa, including a soluble amelogenin based peptide with an amino acid weight between 0, 5-2 kDa, 2-4.5 kDa, 1-5.5 kDa, 1.4-6.5 kDa, 0.5-1.5 kDa, or 0.5-1.3 kDa, that is originally comprised in the eluate represented by the third and/or fourth peak of an HPLC analysis of processed amelogenin.

In general, the enamel matrix, enamel matrix derivatives and enamel matrix proteins are hydrophobic substances, i.e. less soluble in water, especially at increased temperatures. In general, these proteins are soluble at non-physiological pH values and at a low temperature such as about 4-20° C., while they will aggregate and precipitate at body temperature (35-37° C.) and neutral pH. The size of the aggregates is variable, comprising an average of size between about 200-500 nm in diameter, often bigger, such as between 100 nm-1 μM, 200 nm-1 μm, 250 nm-1 μm, 500 nm-1 μm, 500 nm-1 μm, 100-250 nm, 100-500 nm, 200-300 nm, 200-500 nm, 200-600 nm, 250-450 nm, 250 nm-500 nm, 250 nm-750 nm, 500 nm-750 nm, etc. In general, the size of the aggregates can be variable, depending also on the concentration of proteins and/or peptides in a given solution/matrix.

In a presently preferred embodiment, a formulation of the present recombinant enamel matrix proteins, for use according to the present invention, thus comprises active enamel substances which at least partially are aggregated, and/or which after application in vivo are capable of forming aggregates. The particle size of said aggregates being in a range of from about 1 μm to about 20 nm, such as between 1 μm and 20 nm, 1 μm and 10 nm, 5 μm and 10 nm, 10 μm and 1 nm, 100 μm and 10 nm, 100 μm and 1 nm, 1 μm and 1 nm, 1 μm and 5 nm, 1 μm and 15 nm.

In accordance to the present invention, a formulation of a recombinant protein purified according to the invention, such as an enamel matrix protein or a fragment thereof and/or a fusion protein comprising an enamel matrix protein and/or a fusion protein comprising at least a portion derived from an enamel matrix protein, may be used together with other active drug substances such as, e.g. anti-bacterial, anti-inflammatory, antiviral, antifungal substances or in combination with local chemotherapy, inducers of apoptosis, growth factors such as, e.g., TGFβ, PDGF, IGF, FGF, EGF, keratinocyte growth factor or peptide analogues thereof. Enzymes—either inherently present in the enamel matrix or preparation thereof or added—may also be used in combination with an enamel matrix fraction and/or polypeptide fragment according to the present invention, especially proteases.

Consequently, in a presently preferred embodiment, the present invention relates to a process for purifying a recombinant protein or a fragment thereof, wherein the protein is selected from the group consisting of an enamel matrix, enamel matrix derivative and/or an enamel matrix protein. In an equally preferred embodiment, the present invention relates to a process for purifying a recombinant protein wherein the protein is selected from the group consisting of amelogenin, proline-rich non-amelogenins, amelin, tuftelin, tuft proteins, serum proteins, salivary proteins, ameloblastin, sheathlin, and derivatives thereof, and mixtures thereof.

Furthermore, the present invention relates to a process for purifying a recombinant protein wherein the protein is a fusion protein comprising at least a fragment of amelogenin and a fragment of a protein selected from the group consisting of proline-rich non-amelogenins, amelin, tuftelin, tuft proteins, serum proteins, salivary proteins, ameloblastin, sheathlin, and derivatives thereof, and mixtures thereof.

### Recombinant Amelogenin and Amelin

A presently preferred embodiment of the invention relates to synthetic genes encoding the human 175 amino acid amelogenin (without signal peptide) (as seen in SEQ ID NO 1), and the 107 N-terminal amino acids of human amelin (without signal peptide) (as seen in SEQ ID NO 2), which are constructed and expressed in E. coli.

Furthermore, fusion proteins between amelogenin and amelin are also disclosed being expressed in E. coli (as seen in SEQ ID NO 4 and SEQ ID NO 31). Amelogenin and the amelogenin-amelin fusion proteins are produced by the cells. Production of amelogenin and amelogenin-amelin fusion proteins can be carried out in both complex growth mediums (e.g. LB and TB) and in defined medium (e.g. NYAT, M9, M65), and protein expression can be induced by e.g. IPTG and/or lactose.

Consequently, in one particular context, the present invention relates to a process for purifying a recombinant protein, wherein the recombinant protein is amelogenin. The present invention also relates to a process for purifying a recombinant protein, wherein the recombinant protein is encoded by SEQ ID NO 1, and/or comprised in SEQ ID NO 5. Furthermore, the invention relates to a process for purifying a recombinant protein, wherein the recombinant protein is amelin. The present invention also relates to a process for purifying a recombinant protein, wherein the recombinant protein is encoded by SEQ ID NO 2, and/or comprised in SEQ ID NO 6.

Additionally, in a preferred embodiment, the invention relates to a process for purifying a recombinant protein, wherein the protein is a fusion protein comprising at least one fragment of amelogenin. Furthermore, in another embodiment, the invention relates to a process for purifying a recombinant protein, wherein the protein is a fusion protein comprising at least one fragment of amelin. In another context, the invention relates to a process for purifying a recombinant protein wherein the protein is a fusion protein comprising at least one fragment of amelogenin and at least one fragment of amelin.

In another preferred embodiment, the invention relates to a process for purifying a recombinant protein wherein the protein is a fusion protein comprising a C-terminal amino acid sequence originating in amelogenin as shown in SEQ ID NO 5, and/or a N-terminal amino acid sequence originating in amelin, as shown in SEQ ID NO 6, encoded by a sequence comprised in SEQ ID NO 1 and/or in SEQ ID NO 2, respectively. Another preferred embodiment relates to a process for purifying a recombinant protein wherein the protein is a fusion protein comprising an N-terminal amino acid sequence originating in amelogenin as shown in SEQ ID NO 5 and/or a C-terminal amino acid sequence originating in amelin, as shown in SEQ ID NO 6, encoded by a sequence comprised in SEQ ID NO 1 and/or in SEQ ID NO 2, respectively. It will be understood by the skilled artisan, that such fusion proteins according to the invention may comprise any suitable amount of amino acids, herein referring to both the amount of amino acids originating in the amelin part of the fusion protein, and the amount of amino acids originating in the amelogenin part of the fusion protein, which both may be varied independently of each other, wherein said fusion protein is characterized by that it will maintain its original biological function and/or remain a stable protein with an accurate three dimensional structure even after being purified during conditions with a low pH and a high temperature according to a process of the present invention. After a purification process according to the invention, such fusion proteins will maintain their biological function and/or remain stable proteins with an accurate three dimensional structure, either as an intact fusion protein and/or as a protein which after the purification has been cleaved, to generate a functional amelin and/or an amelogenin protein and/or (a) polypeptide fragment(s) thereof. Such a protein will at least retain its activity and/or accurate stability and structure after returning to an environment essentially resembling its natural environment. A fusion protein for use in accordance with the invention comprises from 1-175 amino acids originating in amelogenin and/or from 1-107 amino acids originating in amelin.

In yet another equally preferred embodiment, the invention relates to a process for purifying a recombinant protein, wherein the protein is a amelogenin-amelin fusion protein comprising 175 N-terminal amino acids originating in amelogenin and 107 C-terminal amino acids originating in amelin as shown in SEQ ID NO 4. In yet another embodiment, the invention relates to a process for purifying a recombinant protein wherein the protein is a fusion protein comprising 175 N-terminal amino acids originating in amelogenin and 107 C-terminal amino acids originating in amelin as encoded by the sequence in SEQ ID NO 3. Another preferred embodiment of the present invention relates to a process for purifying a recombinant protein which is an amelin-amelogenin fusion protein comprising 107 N-terminal amino acids originating in amelin and 175 C-terminal amino acid originating in amelogenin according to SEQ ID NO 31 as encoded by the sequence of SEQ ID NO 30.

In a preferred embodiment of the present invention a fusion protein according to the invention is an amelogenin-amelin fusion protein according to SEQ ID NO 4 (encoded by the sequence of SEQ ID NO 3). In another preferred embodiment the fusion protein is a amelin-amelogenin fusion protein according to SEQ ID NO 31 (encoded by the sequence of SEQ ID NO 30).

Additionally, the invention also comprises a fusion protein comprising any suitable amount of amino acids as previously described, wherein the part encoding for amelin and the part encoding for amelogenin in the fusion protein, are divided by a physiological cleavage site designed to suit a particular enzyme, such as, but not limited to, BamHI.

By a “physiological cleavage site” and/or a “cleavage site”, is meant a specific nucleic acid sequence, which is recognized by a specific enzyme, such as a restriction enzyme, and which is then cleaved by said enzyme, to generate two fragments with e.g. cohesive ends, which cohesive ends may be ligated to other fragments with complementary cohesive ends to generate recombinant DNA molecules. Such a cleavage site may be between 2 and 20 nucleotides long, such as between 4 and 8 nucleotides. In the context of the present invention, a nucleic acid construct is generated to produce a construct which encodes a fusion protein wherein amelogenin and amelin both have been cut with a restriction enzyme to generate cohesive ends so that they may be ligated together in said construct. Such a construct is shown in table 5.

It will be clear to the skilled artisan that any physiological cleavage site may be designed and used in a process according to the present invention, to link together nucleic acid fragments which together will encode a fusion protein according to the invention. Examples of such enzymes are any enzymes belonging to the group of nucleases, such as exonucleases and endonucleases. The invention also encompass a process for purifying a recombinant protein, wherein the protein is a fusion protein comprising 107 N-terminal amino acids originating in amelin and 175 C-terminal amino acids originating in amelogenin or a fusion protein comprising 175 N-terminal amino acids originating in amelogenin and 107 C-terminal amino acids originating in amelin divided by a physiological cleavage site between the amelin part and the amelogenin part of the fusion proteins, as shown in table 5. In one preferred embodiment of the invention, such a physiological cleavage site comprises a nucleic acid sequence recognized by the enzyme BamHI.

Furthermore, a fusion protein according to the invention may after the purification process has been completed, in any suitable manner, be separated into a functional amelogenin and/or an amelin protein, and/or into fragments thereof. Said proteins and/or fragments thereof are characterized by that they after the purification process and the separation will maintain their biological function and/or remain a stable protein with an accurate three dimensional structure. Such a separation of a fusion protein will of course also be possible with other fusions proteins as disclosed by the present invention.

As exemplified by the problems that were encountered during expression of amelin and amelin-amelogenin fusion products, it is well known in the field of the art that some proteins and/or peptides are hard/tricky to produce in E. coli, or any other non-secretive host organism, even when their gene is codon optimized. Without wishing to limit the scope of the present invention unduly, the reason for this could be due to a very low stability or unfavourable conformation of the mRNA, or that the protein/peptide in itself is unstable inside the E. coli cell and therefore rapidly degrades.

As is for the first time shown in the experiments accompanying the present application, amelogenin on the other hand is produced at a high level (FIG. 1), both with and without a tag, suggesting that this protein is relatively stable inside the cells. What is more, the amelogenin-amelin fusion is also successfully produced by the cells (FIG. 4), albeit not to exactly as high levels as for amelogenin alone. Thus, a specific aspect of the present invention relates to the use of constructing a fusion protein of the protein and/or peptide of interest to amelogenin to help the expression of other proteins and/or peptides, as exemplified by amelin, maybe by facilitating transcription and/or translation, and/or by stabilizing the protein once expressed. It is easily understandable by the skilled artisan, that other proteins and/or peptides than amelin can be expressed more easily by fusing them to amelogenin.

This might also be facilitated by fusing such proteins and/or peptides of interest to any other thermostable and/or chemostable protein, and/or by fusing such proteins and/or peptides of interest to any other protein that is chemostable and/or solvable at a high temperature and/or low pH.

### Vectors

The present invention also relates to a vector for expressing a recombinant protein of interest, such as a plasmid or a phage, which may be a vector as disclosed by the present invention, or any vector suitable for the indicated purpose. Such a vector is envisioned to be usable for producing a recombinant protein according to a process as described in the present invention.

In a preferred embodiment, such a vector comprises a gene which encodes a protein of interest or a fusion protein. The vector may also incorporate one or more promoters derived from e.g. bacteriophages. Examples of preferred embodiments of such vectors are given in FIG. 6-11.

It is to be understood that the vector examples given in the application are only intended to demonstrate proof of concept of the present invention, and are not intended to limit the scope of the vectors that can be used. A person skilled in the art can easily provide a variety of vectors known in the field of the art today that will be compatible with the process described herein. Other examples of preferred vectors are different pUC derivatives.

The present invention thus in a particular embodiment relates to the vectors pHHAQ (FIG. 10), pQEam (FIG. 11), pHAP (FIG. 6), pETam (FIG. 7), pHAP-am (FIG. 9) and pETam-amg (FIG. 8), as defined later in the experimental section, for expressing a recombinant protein in a host cell. A preferred embodiment of the invention relates to the vectors pHAP-am and pETam-amg. Furthermore, the invention relates to the use of the vectors pHHAQ, pQEam, pHAP, pETam, pHAP-am or pETam-amg, in particular pHAP-am or pETam-amg, for expressing a recombinant protein in a host cell, for use in a process for purifying a recombinant protein as described herein.

Accordingly, another embodiment of the invention encompasses a host cell transfected and/or transformed with a vector selected from pHHAQ, pQEam, pHAP, pETam, pHAP-am or pETam-amg, as defined later in the experimental section, which can be used in a process for purifying a recombinant protein as described herein. A preferred embodiment of the invention relates to a host cell transfected and/or transformed with the vectors pHAP-am or pETam-amg. In yet another embodiment, the invention relates to a purified recombinant protein expressed by a host cell transfected and/or transformed with a vector selected from pHHAQ, pQEam, pHAP, pETam, pHAP-am or pETam-amg, in particular pHAP-am or pETam-amg, per se, optionally purified by a process according to the invention.

In another equally preferred embodiment, the invention refers to a recombinant protein expressed by a host cell transfected and/or transformed with a vector selected from pHHAQ, pQEam, pHAP, pETam, pHAP-am or pETam-amg, in particular pHAP-am or pETam-amg, as defined later in the experimental section, per se, optionally purified by a process according to the invention for use as a medicament.

### Pharmaceutical Compositions

Depending on the use of a recombinant protein purified by a process according to the invention, a composition may be a pharmaceutical and/or a therapeutic and/or a cosmetic composition. In the following the term “a pharmaceutical and/or therapeutic composition” is also intended to embrace cosmetic compositions as well as compositions belonging to the so-called grey area between pharmaceuticals and cosmetics, namely cosmeceuticals.

A pharmaceutical and/or therapeutic composition comprising a recombinant protein purified by a process according to the invention, serves as a drug delivery system. In the present context the term “drug delivery system” denotes a pharmaceutical and/or therapeutic composition (a pharmaceutical and/or therapeutic formulation or a dosage form) that upon administration presents the active substance to the body of a human or an animal.

For the administration to an individual (such as an animal or a human), a recombinant protein purified by a process according to the invention, and/or a preparation thereof, are preferably formulated into a pharmaceutical composition containing the recombinant protein purified by a process according to the invention and, optionally, one or more pharmaceutically acceptable excipients.

A composition comprising a recombinant protein purified by a process according to the invention to be administered, may be adapted for administration by any suitable route, e.g. by systemic administration to a patient through a hose, syringe, spray or draining device.

Furthermore, a composition may be adapted to administration in connection with surgery, e.g. as a systemic administration by infusion into the blood, lymph, ascites, or spinal fluids, or by inhalation. For systemic application, the compositions according to the invention may contain conventionally non-toxic pharmaceutically acceptable carriers and excipients according to the invention, including microspheres and liposomes. Administration of a composition according to the present invention may also be performed via any other conventional administration route, such as, but not limited to, an oral, parenteral, intravenous, buccal, aural, rectal, vaginal, intraperitoneal, topical (dermal), or nasal route, or by the administration to a body cavity such as e.g. a tooth root or a tooth root canal.

Other applications may of course also be relevant such as, e.g., application on dentures, protheses, implants, and application to body cavities such as the oral, nasal and vaginal cavity. The mucosa may be selected from oral, buccal, nasal, aural, rectal and vaginal mucosa. Furthermore, the application may be directly on or onto a wound or other soft tissue injuries.

Furthermore, application within the dental/odontologic area is also of great importance. Relevant examples are application to periodontal (dental) pockets, to gingiva or to gingival wounds or other wounds located in the oral cavity, or in connection with oral surgery.

One especially preferred embodiment of the present invention thus relates to the use a recombinant protein purified by a process described herein for manufacturing a pharmaceutical composition for treating a wound in a gingival tissue, or preventing and/or treating an infection and/or inflammation in a gingival tissue.

It is further anticipated that, due to the antibacterial properties of a recombinant protein purified by a process described herein, it may advantageously be applied to teeth or tooth roots for the prevention of caries and/or plaque. To support this use, it has been shown (Weinmann, J. P. et al: Hereditary disturbances of enamel formation and calcification, J. Amer. Dent. Ass. 32: 397-418, 1945; Sundell S, Hereditary amelogenesis imperfecta. An epidemiological, genetic and clinical study in a Swedish child population, Swed Dent J Suppl 1986; 31: 1-38) that teeth which are imperfectly developed (amelogenesis imperfecta) and consequently contain large amounts of amelogenins are remarkably caries resistant.

A composition for use in accordance with the present invention may be, but is not limited to, in the form of, e.g., a fluid, semi-solid or solid composition such as, but not limited to, dissolved transfusion liquids, such as sterile saline, Ringer's solution, glucose solutions, phosphate buffer saline, blood, plasma, water, powders, microcapsules, bioabsorbable patches, drenches, sheets, bandages, plasters, implants, pills, sprays, soaps, suppositories, vagitories, toothpaste, lotions, mouthwash, shampoo, microspheres, nanoparticles, sprays, aerosols, inhalation devices, solutions, dispersions, wetting agents, suspensions, emulsions, pastes, ointments, hydrophilic ointments, creams, gels, hydrogels, dressings, devices, templates, smart gels, grafts, solutions, emulsions, suspensions, powders, films, foams, pads, sponges (e.g. collagen sponges), transdermal delivery systems, granules, granulates, capsules, agarose or chitosan beads, tablets, microcapsules, freeze-dried powders, granules, granulates or pellets, and mixtures thereof.

Suitable dispersing or wetting agents for use in accordance with the invention, may be naturally occurring phosphatides, e.g., lecithin, or soybean lecithin; condensation products of ethylene oxide with e.g. a fatty acid, a long chain aliphatic alcohol, or a partial ester derivable from fatty acids and a hexitol or a hexitol anhydride, e.g. polyoxyethylene stearate, polyoxyethylene sorbitol monooleate, polyoxyethylene sorbitan monooleate, etc. The invention is however not limited thereto.

Suitable suspending agents are, e.g., naturally occurring gums such as, e.g., gum acacia, xanthan gum, or gum tragacanth; celluloses such as, e.g., sodium carboxymethylcellulose, microcrystalline cellulose (e.g. Avicel® RC 591, methylcellulose); alginates and chitosans such as, but not limited to, sodium alginate, etc.

A liquid composition, for use in accordance with the present invention, may e.g. be, but is not limited to, a solution, dispersion or suspension for application on a surface of e.g. a medical implant or device. Once applied, the composition should preferably solidify, e.g. by drying, to a solid or at least highly viscous composition which does not dissolve on storage or when the implant or device is in use.

Such a composition is preferably applied under sterile conditions and/or sterilised after application by irradiation or exposure to ethylene oxide gas. When the composition is in the form of a liquid composition, it may also be applied shortly before the medical implant or device is to be introduced into the body. As an alternative to applying a composition comprising a recombinant protein purified by a process according to the invention on the medical implant or device, the composition may be applied on a surface of a tissue which is in contact with the implant or device, such as a tissue comprising a substantial proportion of epithelial cells as indicated above. Furthermore, the composition may be applied on both the implant and/or device and on a tissue in contact therewith.

It should also be emphasized that any other pharmaceutical composition as disclosed by the present invention may be used for the application on a surface of a medical implant or device.

A composition according to the present invention, may also, in addition to what already has been disclosed herein, be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988.

A pharmaceutically acceptable excipient is a substance which is substantially harmless to the individual to which the composition is to be administered. An excipient is comprised in a pharmaceutical composition according to the invention. Such an excipient normally fulfils the requirements given by the national health authorities. Official pharmacopoeias such as e.g. the British Pharmacopoeia, the United States of America Pharmacopoeia and The European Pharmacopoeia set standards for pharmaceutically acceptable excipients.

The choice of pharmaceutically acceptable excipient(s) in a composition, and the optimum concentration thereof, for use according to the invention, cannot generally be predicted and must be determined on the basis of an experimental evaluation of the final composition.

However, suitable excipients for the present purpose may be selected from such excipients that promote application of the composition comprising a recombinant protein purified by a process according to the invention on a surface of the implant or device, or that promote the adherence of the composition to the surface on application, or that prevent immediate dissolution of the composition or protract the release of a recombinant protein purified by a process according to the invention from the composition. A person skilled in the art of pharmaceutical formulation can find guidance in e.g., “Remington's Pharmaceutical Sciences”, 18th Edition, Mack Publishing Company, Easton, 1990.

Whether a pharmaceutically acceptable excipient is suitable for use in a pharmaceutical composition is generally dependent on which kind of dosage form is chosen for use for a particular kind of wound, and/or any other type of disorder and/or damage to a body.

The pharmaceutically acceptable excipients may include solvents, buffering agents, preservatives, humectants, chelating agents, antioxidants, stabilizers, emulsifying agents, suspending agents, gel-forming agents, ointment bases, penetration enhancers, perfumes, powders and skin protective agents. It should however be emphasized that the invention is not limited thereto.

Examples of such solvents for use in a composition in accordance with the present invention, are water, alcohols, vegetable or marine oils (e.g. edible oils like almond oil, castor oil, cacao butter, coconut oil, corn oil, cottonseed oil, linseed oil, olive oil, palm oil, peanut oil, poppy seed oil, rape seed oil, sesame oil, soybean oil, sunflower oil, and tea seed oil), mineral oils, fatty oils, liquid paraffin, polyethylene glycols, propylene glycols, glycerol, liquid polyalkylsiloxanes, or other hydrophilic or etheric solvents such as weak acids with a pH of about 5.5-6.0 facilitating the subsequent application of filling materials in the tooth, as well as mixtures thereof.

Examples of buffering agents are citric acid, acetic acid, tartaric acid, lactic acid, hydrogen phosphoric acid, bicarbonates, phosphates, diethylamine etc.

Suitable examples of preservatives are parabens, such as methyl, ethyl, propyl p-hydroxybenzoate, butylparaben, isobutylparaben, isopropylparaben, potassium sorbate, sorbic acid, benzoic acid, methyl benzoate, phenoxyethanol, bronopol, bronidox, MDM hydantoin, iodopropynyl butylcarbamate, EDTA, benzalconium chloride, and benzylalcohol, or mixtures of preservatives.

Examples of humectants are glycerin, propylene glycol, sorbitol, lactic acid, urea, and mixtures thereof.

Examples of chelating agents are sodium EDTA and citric acid.

Examples of antioxidants are butylated hydroxy anisole (BHA), ascorbic acid and derivatives thereof, tocopherol and derivatives thereof, cysteine, and mixtures thereof.

Examples of emulsifying agents are naturally occurring gums, e.g. gum acacia or gum tragacanth; naturally occurring phosphatides, e.g. soybean lecithin, sorbitan monooleate derivatives; wool fats; wool alcohols; sorbitan esters; monoglycerides; fatty alcohols; fatty acid esters (e.g. triglycerides of fatty acids); and mixtures thereof.

Examples of suspending agents are e.g. celluloses and cellulose derivatives such as, e.g., carboxymethyl cellulose, hydroxyethylcellulose, hydroxypropylcellulose, hydroxypropylmethylcellulose, microcrystalline cellulose, carraghenan, acacia gum, arabic gum, tragacanth, and mixtures thereof.

Examples of gel bases, viscosity-increasing agents or components which are able to take up exudate from a wound are: liquid paraffin, polyethylene, fatty oils, colloidal silica or aluminium, zinc soaps, glycerol, propylene glycol, tragacanth, carboxyvinyl polymers, magnesium-aluminium silicates, Carbopol®, hydrophilic polymers such as, e.g. starch or cellulose derivatives such as, e.g., carboxymethylcellulose, hydroxyethylcellulose and other cellulose derivatives, water-swellable hydrocolloids, carragenans, hyaluronates (e.g. hyaluronate gel optionally containing sodium chloride), collagen, gelatine, pectin, chitosans and alginates including propylene glycol aginate.

In the present invention, a recombinant protein purified by a process according to the invention can be incorporated into a polymeric matrix so that it is released by degradation of the polymeric matrix, by enzymatic action and/or by diffusion. Said polymeric matrix is either suitable for cellular in-growth, or cell-occlusive. Comprised in the invention is thus in particular a pharmaceutical and/or cosmetic formulation of an active enamel substance at a low total concentration within the formulation, wherein a spatial and/or selective regulation of release of said recombinant protein permits a great percentage of the active enamel substance to be released at the time of appropriate cellular activity.

Consequently, one aspect of the present invention relates to a pharmaceutical and/or therapeutic formulation for administering a recombinant protein purified by a process according to the invention, comprising a polymeric matrix, either suitable for cellular growth, in-growth and/or migration, or being cell-occlusive, and a recombinant protein, wherein said matrix is formed by a nucleophilic addition reaction between a strong nucleophile and a conjugated unsaturated bond, or a conjugated unsaturated group.

Preferably, the conjugated unsaturated groups or conjugated unsaturated bonds are acrylates, vinylsulfones, methacrylates, acrylamides, methacrylamides, acrylonitriles, vinylsulfones, 2- or 4-vinylpyridinium, maleimides, or quinones.

Examples of ointment bases are e.g. beeswax, paraffin, cetanol, cetyl palmitate, vegetable oils, sorbitan esters of fatty acids (Span), polyethylene glycols, and condensation products between sorbitan esters of fatty acids and ethylene oxide, e.g. polyoxyethylene sorbitan monooleate (Tween).

Examples of hydrophobic or water-emulsifying ointment bases are paraffins, vegetable oils, animal fats, synthetic glycerides, waxes, lanolin, and liquid polyalkylsiloxanes.

Examples of hydrophilic ointment bases are solid macrogols (polyethylene glycols).

Other examples of ointment bases are triethanolamine soaps, sulphated fatty alcohol and polysorbates.

Examples of powder components are: alginate, collagen, lactose, powder which is able to form a gel when applied to a wound (absorbs liquid/wound exudate). Normally, powders intended for application on large open wounds must be sterile and the particles present must be micronized.

Examples of other excipients are polymers such as carmelose, sodium carmelose, hydroxypropylmethylcellulose, hydroxyethylcellulose, hydroxypropylcellulose, methylcellulose, pectin, xanthan gum, locust bean gum, acacia gum, gelatin, carbomer, emulsifiers like vitamin E, glyceryl stearates, cetanyl glucoside, collagen, carrageenan, hyaluronates and alginates and kitosans.

Examples of diluents and disintegrating agents are but not limited to lactose, saccharose, emdex, calcium phosphates, calcium carbonate, calcium sulphate, mannitol, starches and microcrystalline cellulose.

Examples of binding agents are, but not limited to, saccharose, sorbitol, gum acacia, sodium alginate, gelatine, starches, cellulose, sodium coboxymethylcellulose, methylcellulose, hydroxypropylcellulose, polyvinylpyrrolidone and polyetyleneglycol.

Compositions which have proved to be of importance in connection with topical application are those which have tixothropic properties, i.e. the viscosity of the composition is affected e.g. by shaking or stirring so that the viscosity of the composition at the time of administration can be reduced and when the composition has been applied, the viscosity increases so that the composition remains at the application site.

However, it is appreciated that in those cases where a pharmaceutically acceptable excipient may be employed in different dosage forms or compositions, the application of a particular pharmaceutically acceptable excipient is not limited to a particular dosage form or of a particular function of the excipient.

The application of a composition comprising a recombinant protein purified by a process according to the invention, is intended for inducing and stimulating connective tissue cell invasion, proliferation and growth as part of a repair process following cytoreductive surgery, significant removal or loss of tissue. Other applications may of course also be relevant, such as application directly in or into a deep wound, or other substantial tissue defects.

Dressings and/or bandages are also important delivery systems for a recombinant protein purified by a process according to the invention. When dressings are used as dosage form, a recombinant protein purified by a process according to the invention may be admixed with the other material/ingredients before or during the manufacture of the dressing or, the recombinant protein may in some way be coated onto the dressing e.g. by dipping the dressing in a solution or dispersion of the a recombinant protein, or by spraying a solution or dispersion of a recombinant protein onto the dressing. Alternatively, a recombinant protein may be applied in the form of a powder to the dressing. Dressings may be in the form of absorbent wound dressings for application to exuding wounds. Dressings may also be in the form of hydrogel dressings (e.g. cross-linked polymers such as, e.g. Intrasites which contains carboxymethylcellulose, propylene glycol or polysaccharide, disaccharide and proteins) or in the form of occlusive dressings such as, e.g., alginates, chitosan, hydrophilic polyurethane film, collagen sheets, plates, powders, foams, or sponges, foams (e.g. polyurethane or silicone), hydrocolloids (e.g. carboxymethylcellulose, CMC), collagen and hyaluronic acid-based dressings including combinations thereof.

Alginate, chitosan and hydrocolloid dressings take up wound exudate when placed on a wound. When doing so they produce an aqueous gel on the surface of the wound and this gel is believed to be beneficial for the healing of the wound due to the retaining of moisture in the wound.

It is also envisaged that the a recombinant protein purified by a process according to the invention, may be incorporated in a tissue adhesive also comprising, e.g. fibrinogen and thrombin and optionally Factor XIII or another plasma coagulation factor to provide hemostasis. The tissue adhesive may either be prepared as a premix of a recombinant protein, fibrinogen and optionally Factor XIII, thrombin being added to the premix immediately before the tissue adhesive is applied on the wound. Alternatively, the premix of fibrinogen and a recombinant protein purified by a process according to the invention, and optionally Factor XIII may be applied on the wound before application of thrombin. In situ, the thrombin converts fibrinogen to fibrin thereby reproducing the coagulation process occurring naturally in wound healing. The presence of the recombinant protein in the tissue adhesive may serve to accelerate the wound healing process as discussed above. A commercial product which may be used for inclusion of the a recombinant protein purified by a process according to the invention is Tisseel®, a two-component fibrin sealant produced by Immuno, AG, Vienna, Austria.

In a toothpaste or mouthwash formulation or other formulation for application to teeth or tooth roots, a recombinant protein purified by a process according to the invention may either be present in a dissolved state in a vehicle of slightly acid pH or as a dispersion in a vehicle of neutral pH. It is anticipated that in use a recombinant protein purified by a process according to the invention may form a protective layer on the surface of the teeth, thereby preventing the attachment of caries producing bacteria (cf. Example 4 below). In such dental care preparations, a recombinant protein may be formulated together with one or more other compounds which have a caries preventive effect, notably fluorine or another trace element such as vanadium or molybdenum. At neutral pH, the trace element is believed to be bound to (e.g. by ion bonds) or embedded in the active enamel substance from which it is released to exert its caries preventive effect when a recombinant protein is dissolved at a pH of about 5.5 or less, e.g. due to acid production by caries producing bacteria.

In a pharmaceutical composition for use according to the invention, a recombinant protein purified by a process according to the invention is generally present in a concentration ranging from about 0.01% to about 99.9% w/w. The amount of composition applied will normally result in an amount of total protein per cm² area of dental pulp corresponding to from about 0.005 mg/mm² to about 5 mg/mm² such as from about 0.01 mg/mm² to about 3 mg/mm².

In those cases where the a recombinant protein purified by a process according to the invention is administered in the form of a liquid composition, the concentration of the recombinant protein in the composition is in a range corresponding to from about 0.01 to about 50 mg/ml, e.g. from about 0.1 to about 30 mg/ml. Higher concentrations are in some cases desirable and can also be obtained such as a concentration of at least about 100 mg/ml.

Defect areas in dental pulp in humans typically have a size of about 5-10×2-4×5-10 mm corresponding to about 200 μl and normally at the most about 0.5-1 ml such as about 0.2-0.3 ml per tooth is applied of a composition having a concentration of about 1-40 mg total protein/ml such as, e.g., 5-30 mg/ml is applied. 0.2-0.3 mg/ml corresponds to about 6 mg protein per 25-100 mm² or about 0.1 mg/mm² if calculated only on root surface. Normally an excessive volume is applied to cover the affected surfaces adequately. Even a multilayer would only require a small fraction of the above-mentioned amounts.

One preferred embodiment of the present invention comprises a pharmaceutical composition comprising a recombinant enamel matrix protein, such as amelogenin, or a fragment thereof and/or a fusion protein comprising at least a portion of an enamel matrix protein, such as amelogenin, purified by a process according to the invention.

### LEGENDS TO FIGURES

FIG. 1.

Total cell proteins analysed by SDS-PAGE for cells expressing untagged amelogenin from pHAP (A) or tagged amelogenin from pHHAQ (B) compared to control cells (C). Cells were grown in LB-medium and TB-medium. Amelogenin band is indicated with an arrow.

FIG. 2.

SDS-PAGE gel showing untreated cells expressing amelogenin from pHAP (U), grown in LB-medium, and protein extracts of the same cells subjected to sonication (Sonic.) followed by heat treatment at 80° C. (Sonic.+Heat). The cells were disrupted and heat treated in Buffer A (A), 0.1% HAc (0.1), or 3% HAc (0.3). The amelogenin bands are indicated with an arrow.

FIG. 3.

SDS-PAGE gel showing untreated amelogenin expressing cells harbouring the vector pHAP (U), grown in LB-medium, and samples from different steps in amelogenin purification. The cells were disrupted by sonication in 3% HAc and the soluble fraction (S) was then subjected to a heat treatment at 80° C. (S+80). One sample of untreated cells was subjected to the heat treatment without prior disruption (80). The amelogenin bands are indicated with an arrow.

FIG. 4.

Analysis by SDS-PAGE (A) and Western blot (B) of cells expressing the amelogenin-amelin fusion protein from the pHAP-am expression vector. Samples were taken from cells harbouring pHAP-am 2, 4 and 21 hours after induction, and for uninduced cells (UI). The samples were compared to control cells (C) harbouring empty pET11a vector. The bands corresponding to the fusion protein are indicated with an arrow.

FIG. 5.

SDS-PAGE of purified samples of human amelogenin (pHAP) and amelogenin-amelin fusion protein (pHAP-am). The cells expressing the recombinant proteins have been grown in different growth mediums (LB, TB or NYAT), and protein expression has been induced with either IPTG (I) or lactose (L).

FIG. 6.

pHAP. Plasmid map of insert in pET 11a (Novagen). The beginning and end of the amelogenin coding sequence are shown in italics and the start/stop codons are shown in bold text. Restriction enzyme sites are underlined.

FIG. 7.

pETam. Plasmid map of insert in pET 11a (Novagen). The beginning and end of the amelin coding sequence are shown in italics and the start/stop codons are shown in bold text. Restriction enzyme sites are underlined.

FIG. 8.

pETam-amg. Plasmid map of insert in pET 11a (Novagen). The beginning and end of the amelogenin/amelin coding sequence are shown in italics and the start/stop codons are shown in bold text. Restriction enzyme sites are underlined.

FIG. 9.

pHAP-am. Plasmid map of insert in pET 11a (Novagen). The beginning and end of the amelogenin/amelin coding sequence are shown in italics and the start/stop codons are shown in bold text. Restriction enzyme sites are underlined.

FIG. 10.

pHHAQ. Plasmid map of insert in pQE-30 (Qiagen). The beginning and end of the amelogenin coding sequence is shown in italics and the start/stop codons are shown in bold text. Restriction enzyme sites are underlined. Also shown is N-terminal tag, the MRGSH₆GS epitope.

FIG. 11.

pQEam. Plasmid map of insert in pQE-30 (Qiagen). The beginning and end of the amelin coding sequence is shown in italics and the start/stop codons are shown in bold text. Restriction enzyme sites are underlined. Also shown is N-terminal tag, the MRGSH₆GS epitope.

### EXPERIMENTAL SECTION

### Materials & Methods

Synthesis of a Gene Encoding the 175 Amino Acid Human Amelogenin and Cloning into Expression Vectors

A gene encoding the human 175 amino acid (aa) amelogenin (X-chromosome, without signal peptide) was designed, using the preferred codons for Escherichia coli, and synthesized using polymerase chain reaction (PCR). The gene was constructed using 9 overlapping oligonucleotides between 72-78 bases in length (Table 1).

The oligonucleotides were mixed in equal amounts to a concentration of 5 μM/oligo nucleotide, and to assemble the amelogenin gene 0.5 μl of the oligonucleotide mixture was used to a 50 μl PCR reaction utilising Pfu Ultra Hotstart DNA polymerase (Stratagene). The gene assembly started with 2 minutes of denaturation at 95° C., followed by 30 cycles of the following program: 95° C. for 30 seconds, 54° C. for 30 seconds, and 72° C. for 1 minute. The assembly ended with 10 minutes of elongation for 10 minutes at 95° C.

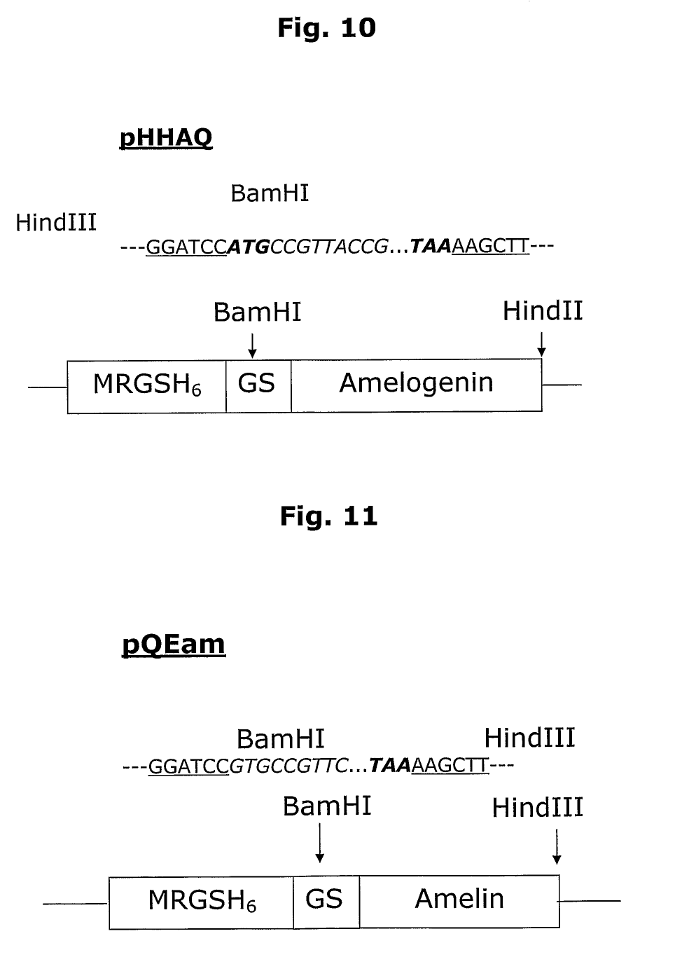

To amplify the assembled gene 3 μl of the mix was used as template in an additional PCR using primers HAX175forw and HAX175rev (Table 1). The primers contained sites for restriction enzymes BamHI and HindIII (underlined), and the amelogenin gene was amplified using the same PCR program as for the assembly PCR. The PCR-product was purified using QIAquick PCR Purification Kit (Qiagen), and subsequently double digested with BamHI and HindIII (Fermentas). The cleavage products were separated on an agarose gel, and the amelogenin band was excised and purified using QIAquick Gel Extraction Kit (Qiagen). The amelogenin fragment was then ligated into the plasmid pQE-30 (Qiagen), previously linearized with BamHI and HindIII. The ligation mixture was used to transform competent E. coli TG1 cells, and the transformants were checked for the amelogenin insert with colony PCR using the primers HAX175forw and HAX175 rev. Plasmids were prepared, using QIAprep Spin Miniprep Kit (QIAgen), from transformants that showed an amelogenin band of corrects size, and the plasmids were sequenced to verify the nucleotide sequence. The transformants showed around five errors in the sequences, compared to the designed sequence, containing deletions, insertions and base substitutions. The sequence was corrected using QuikChange Site-Directed Mutagenesis Kit (Stratagene). The final construct, generating the human 175 aa amelogenin with the N-terminal tag MRGSHHHHHHGS (SEQ ID NO 32), was verified by sequencing and named pHHAQ (FIG. 10).

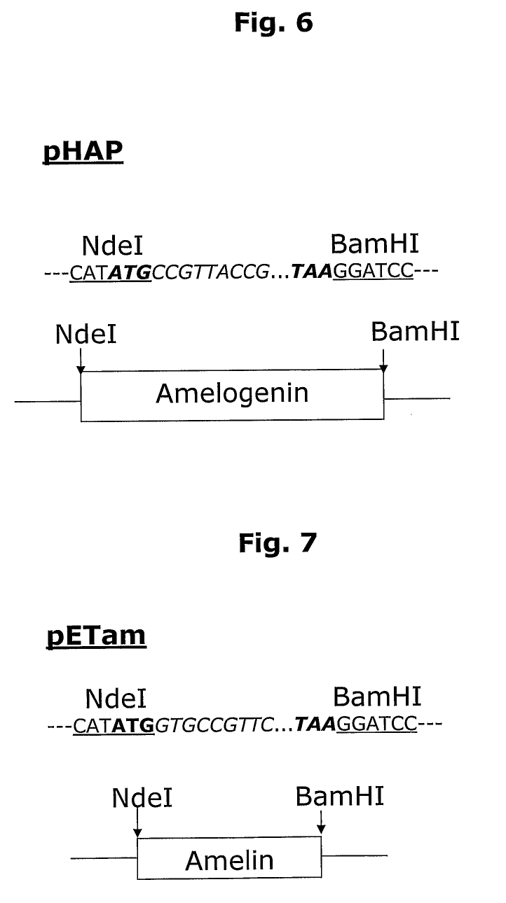

To generate an expression vector for untagged amelogenin, the gene encoding human 175 aa amelogenin was cloned into the plasmid pET11a (Novagen). The plasmid pHHAQ was used as template in a PCR-reaction using PfuUltra Hotstart DNA polymerase. The primers HAXpETforw and HAXpETrev (Table 1) that were used for the amplification contain sites for restriction enzymes NdeI and BamHI (underlined). The PCR-program was the same as before, and the PCR products were purified as above and double digested with NdeI and BamHI (Fermentas). The cleavage products were separated on an agarose gel and the amelogenin fragment was extracted as above, and subsequently ligated into pET11a, linearized with NdeI and BamHI. The ligation mixture was used to transform competent E. coli TG1 and the transformants were analyzed with colony PCR. Plasmids from positive clones were prepared as above and DNA sequencing was used for verification. The construct with the human 175 aa amelogenin cloned into pET11a was named pHAP (FIG. 6), and was subsequently used to transform competent E. coli BL21(DE3) (Novagen).

The synthetic nucleotide sequence encoding the 175 aa human amelogenin is found in Table 2, together with the corresponding amino acid sequence.

TABLE 1
Oligo nucleotides and primers used for synthesis
and cloning of human 175 amino acid amelogenin.
Restriction sites are underlined.

| Oligo | |
|---|---|
| nucleotide/Primer | Sequence 5′ to 3′ |
| AMGO1 | ATGCCGTTACCGCCACACCCGGGTCATCCAGG |
| (SEQ ID NO 7) | TTATATCAACTTCTCTTACGAGGTCTTGACCC |
| | CTTTAAAATGGTAC |
| AMGO2 | AACCATCCACCCATAGGTTCATAGCCATAACT |
| (SEQ ID NO 8) | AGGATACGGAGGACGGATGGATTGGTACCATT |
| | TTAAAGGGGTCAAG |
| AMGO3 | GAACCTATGGGTGGATGGTTGCACCATCAGAT |
| (SEQ ID NO 9) | TATCCCTGTTTTAAGTCAGCAACACCCGCCCA |
| | CTCATACATTACAG |
| AMGO4 | TTGGCTGTTGAGGAATAACCGGCTGTTGGGCA |
| (SEQ ID NO 10) | GGTACCACAGGAATGTGATGGTGAGGCTGTAA |
| | TGTATGAGTGGGCG |
| AMGO5 | CGGTTATTCCTCAACAGCCAATGATGCCAGTG |
| (SEQ ID NO 11) | CCGGGTCAGCATTCTATGACCCCAATTCAACA |
| | TCATCAGCCTAA |
| AMGO6 | TGGCTGCGGTTGTACGGGTTGGGGTTGGTACG |
| (SEQ ID NO 12) | GCTGTTGTGCAGGTGGCGGTAAATTAGGCTGA |
| | TGATGTTGAATTGG |
| AMGO7 | AACCCGTACAACCGCAGCCACATCAACCGATG |
| (SEQ ID NO 13) | CAGCCGCAGCCGCCAGTGCATCCGATGCAGCC |
| | TCTGCCACCTCAAC |
| AMGO8 | GTCAAATCCGGCAACATCGGTGGAAGCGGTTG |
| (SEQ ID NO 14) | CATAGGAAACATTGGCGGTAATGGCGGTTGAG |
| | GTGGCAGAGGCTGC |
| AMGO9 | CCGATGTTGCCGGATTTGACGCTGGAAGCCTG |
| (SEQ ID NO 15) | GCCCTCTACGGATAAAACCAAACGTGAAGAGG |
| | TTGACTAA |
| HAX175forw | AAAGCGGGATCCATGCCGTTACCGCCACACCC |
| (SEQ ID NO 16) | |
| HAX17rev | TTTGCGAAGCTTTTAGTCAACCTCTTCACGTT |
| (SEQ ID NO 17) | TGG |
| HAXpETforw | GTGTCCAGCGCATATGCCGTTACCGCCACACC |
| (SEQ ID NO 18) | C |
| HAXpETrev | CAACGGTGCCGGATCCTTAGTCAACCTCTTCA |
| (SEQ ID NO 19) | CGTTTGG |

TABLE 2
Nucleotide sequence and amino acid sequence for
175 amino acid human amelogenin.

| | |
|---|---|
| Nucleotide sequence | ATG CCG TTA CCG CCA CAC CCG GGT |
| of amelogenin | CAT CCA GGT TAT ATC AAC TTC TCT |
| (SEQ ID NO 1) | TAC GAG GTC TTG ACC CCT TTA AAA |
| | TGG TAC CAA TCC ATC CGT CCT CCG |
| | TAT CCT AGT TAT GGC TAT GAA CCT |
| | ATG GGT GGA TGG TTG CAC CAT CAG |
| | ATT ATC CCT GTT TTA AGT CAG CAA |
| | CAC CCG CCC ACT CAT ACA TTA CAG |
| | CCT CAC CAT CAC ATT CCT GTG GTA |
| | CCT GCC CAA CAG CCG GTT ATT CCT |
| | CAA CAG CCA ATG ATG CCA GTG CCG |
| | GGT CAG CAT TCT ATG ACC CCA ATT |
| | CAA CAT CAT CAG CCT AAT TTA CCG |
| | CCA CCT GCA CAA CAG CCG TAC CAA |
| | CCC CAA CCC GTA CAA CCG CAG CCA |
| | CAT CAA CCG ATG CAG CCG CAG CCG |
| | CCA GTG CAT CCG ATG CAG CCT CTG |
| | CCA CCT CAA CCG CCA TTA CCG CCA |
| | ATG TTT CCT ATG CAA CCG CTT CCA |
| | CCG ATG TTG CCG GAT TTG ACG CTG |
| | GAA GCC TGG CCC TCT ACG GAT AAA |
| | ACC AAA CGT GAA GAG GTT GAC TAA |
| Amino acid sequence | MPLPPHPGHPGYINFSYEVLTPLKWYQSIRP |
| of amelogenin | PYPSYGYEPMGGWLHHQIIPVLSQQHPPT |
| (SEQ ID NO 5) | HTLQPHHHIPVVPAQQPVIPQQPMMPVPGQH |
| | SMTPIQHHQPNLPPPAQQPYQPQPVQPQP |
| | HQPMQPQPPVHPMQPLPPQPPLPPMFPMQPL |
| | PPMLPDLTLEAWPSTDKTKREEVD- |

Synthesis of a Gene Encoding the N-Terminal of Human Amelin and Cloning into Expression Vectors

A gene encoding the 107 N-terminal amino acids (without the signal peptide) of the human amelin was designed according to the preferred codons of E. coli and synthesized using PCR. The gene was assembled by using six overlapping oligonucleotides between 69-72 nucleotides in length (Table 3). The assembly PCR was carried out with 0.5 μl oligo mix (5 μM of each oligo nucleotide) in a 50 μl reaction. Pfu Ultra Hotstart DNA polymerase was used and the following PCR-program was applied for 30 cycles: 95° C. for 30 seconds, 50° C. for 30 seconds and 72° C. for 45 seconds. Before the first cycle an extra denaturation step (95° C. for 5 minutes) was carried out, and also an extra elongation step after the last cycle (72° C. for 10 minutes). The amelin fragment was amplified using the assembly mixture as template and the primers Am-forw and Am-rev, containing restriction sites for BamHI and HindIII (Table 3). The same PCR-program as above was used, except that only 25 cycles was performed, and the annealing temperature was increased to 55° C. The PCR-product was purified and the amelin fragment, was digested with BamHI and HindIII and cloned into pQE-30 as described for the human amelogenin. One of the obtained clones was found to have the correct sequence when analysed by DNA sequencing, and was named pQEam (FIG. 11).

The amelin fragment was also cloned into the pET11a vector by using pQEam as template, and the primers Am-pETforw and Am-pETrev (Table 3). The amplification was carried out using the same 25-cycle PCR-program as above, and the amelin fragment was subsequently purified, digested and ligated into pET11a in the same manner as for the human amelogenin. The construct was verified by sequencing and was finally transformed into E. coli BL21 (DE3). The construct was named pETam (FIG. 7).

The synthetic nucleotide sequence encoding the N-terminal 107 amino acids (without the signal peptide) of human amelin is shown together with the amino acid sequence in Table 4.

TABLE 3
Oligonucleotides and primers used for synthesis
and cloning of the 107 N-terminal aa of human
amelin. Restriction sites are underlined.

| Oligo | |
|---|---|
| nucleotide/Primer | Sequence 5′ to 3′ |
| AO1 | GTGCCGTTCTTTCCGCAGCAAAGCGGTACCCC |
| (SEQ ID NO 20) | AGGCATGGCATCGCTGAGCCTGGAGACCATGC |
| | GCCAGT |
| AO2 | AGCCATAACGACTATATTGCGACAGCGTGTTC |
| (SEQ ID NO 21) | AGGCGCTGTAAACTACCTAACTGGCGCATGGT |
| | CTCCAGG |
| AO3 | TCGCAATATAGTCGTTATGGCTTCGGCAAATC |
| (SEQ ID NO 22) | TTTTAATTCCTTGTGGATGCATGGCCTGTTGC |
| | CGCCACAC |
| AO4 | TTCGTATTGCTGGGTTTCATGTTCGCGAGGAC |
| (SEQ ID NO 23) | GCATCCAAGGCAGGCTAGAGTGTGGCGGCAAC |
| | AGGCCAT |
| AO5 | ACATGAAACCCAGCAATACGAATACTCCTTGC |
| (SEQ ID NO 24) | CTGTTCACCCACCGCCGCTGCCGTCCCAGCCG |
| | AGCCTGAA |
| AO6 | CGTCGCGGCCGCGCTCTGTAAAAACGGTTTCA |
| (SEQ ID NO 25) | GACCCGGTTGCTGCGGTTTCAGGCTCGGCTGG |
| | GACGG |
| Am-forw | TAGCCGGGATCCGTGCCGTTCTTTCCGCAGCA |
| (SEQ ID NO 26) | |
| Am-rev | ATGGCGAAGCTTTTACGTCGCGGCCGCGCTCT |
| (SEQ ID NO 27) | GTA |
| Am-pETforw | GTGTCCAGCGCATATGGTGCCGTTCTTTCCGC |
| (SEQ ID NO 28) | AGCA |
| Am-pETrev | CAACGGTGCCGGATCCTTACGTCGCGGCCGCG |
| (SEQ ID NO 29) | CTCTGTA |

TABLE 4
Nucleotide sequence and amino acid sequence of the
107 N-terminal amino acids of human amelin
(without the signal peptide).

| | |
|---|---|
| Nucleotide sequence | GTG CCG TTC TTT CCG CAG CAA AGC |
| of human amelin | GGT ACC CCA GGC ATG GCA TCG CTG |
| (107 N-terminal aa) | AGC CTG GAG ACC ATG CGC CAG TTA |
| (SEQ ID NO 2) | GGT AGT TTA CAG CGC CTG AAC ACG |
| | CTG TCG CAA TAT AGT CGT TAT GGC |
| | TTC GGC AAA TCT TTT AAT TCC TTG |
| | TGG ATG CAT GGC CTG TTG CCG CCA |
| | CAC TCT AGC CTG CCT TGG ATG CGT |
| | CCT CGC GAA CAT GAA ACC CAG CAA |
| | TAC GAA TAC TCC TTG CCT GTT CAC |
| | CCA CCG CCG CTG CCG TCC CAG CCG |
| | AGC CTG AAA CCG CAG CAA CCG GGT |
| | CTG AAA CCG TTT TTA CAG AGC GCG |
| | GCC GCG ACG TAA |
| Amino acid sequence | VPFFPQQSGT PGMASLSLET |
| of the N-terminal | MRQLGSLQRL NTLSQYSRYG |
| of human amelin | FGKSFNSLWM HGLLPPHSSL |
| (SEQ ID NO 6) | PWMRPREHET QQYEYSLPVH |
| | PPPLPSQPSL KPQQPGLKPF LQSAAAT- |

### Construction of Fusion Proteins

Two different fusion protein constructs were created in the vector pET11a: amelogenin-amelin and amelin-amelogenin. The amelogenin fragment used was the 175 aa human amelogenin synthesized above, and the amelin fragment used was the N-terminal 107 aa of the human amelin described above. Both fusions have a short glycine-serine linker between the amelogenin and amelin parts, corresponding to a BamHI restriction site in the nucleotide sequence.

The amelogenin fragment used for the amelin-amelogenin fusion was amplified from pHAP by using the primers HAX175forw and HAXpETrev, both containing a BamHI site. Primers Am-forw and Am-pETrev, also containing BamHI sites, were used for amplification of the amelin fragment, from pETam, for construction of the amelogenin-amelin fusion. The amplified amelogenin and amelin fragments were purified and digested with BamHI. The cleavage products were separated on an agarose gel and extracted from the gel as before.

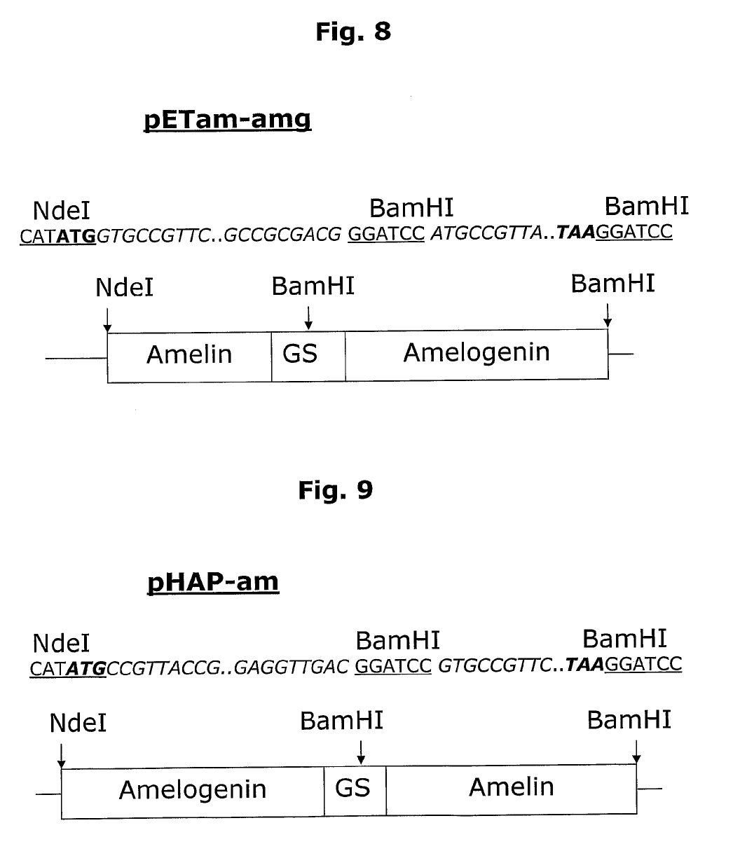

The amelogenin and amelin fragments were ligated into the BamHI site of pETam and pHAP, respectively. The stop codons of pETam and pHAP had previously been removed using QuikChange Site-Directed Mutagenesis Kit, and the plasmids had been linearized with BamHI and dephosphorylated with calf intestine alkaline phosphatase (Roche). The ligation mixtures were used to transform competent E. coli TG1 cells and the colonies were checked for insert presence and orientation by colony PCR. Positive clones were analysed with DNA sequencing and correct sequences for both constructs were found. The amelogenin-amelin construct, named pHAP-am (FIG. 9), and the amelin-amelogenin construct, named pETam-amg (FIG. 8), were both transformed into E. coli BL21 (DE3).

The nucleotide sequences encoding the amelogenin-amelin fusion proteins are seen in Table 5, together with the corresponding amino acid sequences.

TABLE 5
Nucleotide sequence encoding the fusions of
175 aa human amelogenin and 107 N-terminal aa of
human amelin. Between the amelogenin and amelin
sequences is a short glycine-serine linker corres-
ponding to a BamHI restriction site (underlined).

| | |
|---|---|
| Nucleotide | ATG CCG TTA CCG CCA CAC CCG GGT |
| sequence of | CAT CCA GGT TAT ATC AAC TTC TCT |
| amelogenin-amelin | TAC GAG GTC TTG ACC CCT TTA AAA |
| fusion protein | TGG TAC CAA TCC ATC CGT CCT CCG |
| (SEQ ID NO 3) | TAT CCT AGT TAT GGC TAT GAA CCT |
| | ATG GGT GGA TGG TTG CAC CAT CAG |
| | ATT ATC CCT GTT TTA AGT CAG CAA |
| | CAC CCG CCC ACT CAT ACA TTA CAG |
| | CCT CAC CAT CAC ATT CCT GTG GTA |
| | CCT GCC CAA CAG CCG GTT ATT CCT |
| | CAA CAG CCA ATG ATG CCA GTG CCG |
| | GGT CAG CAT TCT ATG ACC CCA ATT |
| | CAA CAT CAT CAG CCT AAT TTA CCG |
| | CCA CCT GCA CAA CAG CCG TAC CAA |
| | CCC CAA CCC GTA CAA CCG CAG CCA |
| | CAT CAA CCG ATG CAG CCG CAG CCG |
| | CCA GTG CAT CCG ATG CAG CCT CTG |
| | CCA CCT CAA CCG CCA TTA CCG CCA |
| | ATG TTT CCT ATG CAA CCG CTT CCA |
| | CCG ATG TTG CCG GAT TTG ACG CTG |
| | GAA GCC TGG CCC TCT ACG GAT AAA |
| | ACC AAA CGT GAA GAG GTT GAC |
| | GGATCC |
| | GTG CCG TTC TTT CCG CAG CAA AGC |
| | GGT ACC CCA GGC ATG GCA TCG CTG |
| | AGC CTG GAG ACC ATG CGC CAG TTA |
| | GGT AGT TTA CAG CGC CTG AAC ACG |
| | CTG TCG CAA TAT AGT CGT TAT GGC |
| | TTC GGC AAA TCT TTT AAT TCC TTG |
| | TGG ATG CAT GGC CTG TTG CCG CCA |
| | CAC TCT AGC CTG CCT TGG ATG CGT |
| | CCT CGC GAA CAT GAA ACC CAG CAA |
| | TAC GAA TAC TCC TTG CCT GGT CAC |
| | CCA CCG CCG CTG CCG TCC CAG CCG |
| | AGC CTG AAA CCG CAG CAA CCG GGT |
| | CTG AAA CCG TTT TTA CAG AGC GCG |
| | GCC GCG ACG TAA |
| Amino acid | MPLPPHPGHPGYINFSYEVLTPLKWYQSIRP |
| sequence of | PYPSYGYEPMGGWLHHQIIPVLSQQHPPTHT |
| amelogenein-amelin | LQPHHHIPVVPAQQPVIPQQPMMPVPGQHSM |
| fusion protein | TPIQHHQPNLPPPAQQPYQPQPVQPQPHQPM |
| (SEQ ID NO 4) | QPQPPVHPMQPLPPQPPLPPMFPMQPLPPML |
| | PDLTLEAWPSTDKTKREEVDGS |
| | VPFFPQQSGT PGMASLSLET |
| | MRQLGSLQRL NTLSQYSRYG |
| | FGKSFNSLWM HGLLPPHSSL |
| | PWMRPREHET QQYEYSLPVH |
| | PPPLPSQPSL KPQQPGLKPF LQSAAAT- |
| Nucleotide | ATG GTG CCG TTC TTT CCG CAG CAA |
| sequence of | AGC GGT ACC CCA GGC ATG GCA TCG |
| amelin-amelogenin | CTG AGC CTG GAG ACC ATG CGC CAG |
| fusion protein | TTA GGT AGT TTA CAG CGC CTG AAC |
| (SEQ ID NO 30) | ACG CTG TCG CAA TAT AGT CGT TAT |
| | GGC TTC GGC AAA TCT TTT AAT TCC |
| | TTG TGG ATG CAT GGC CTG TTG CCG |
| | CCA CAC TCT AGC CTG CCT TGG ATG |
| | CGT CCT CGC GAA CAT GAA ACC CAG |
| | CAA TAC GAA TAC TCC TTG CCT GGT |
| | CAC CCA CCG CCG CTG CCG TCC CAG |
| | CCG AGC CTG AAA CCG CAG CAA CCG |
| | GGT CTG AAA CCG TTT TTA CAG AGC |
| | GCG GCC GCG ACG |
| | GGATCC |
| | ATG CCG TTA CCG CCa CAC CCG GGT |
| | CAT CCa GGT TAT ATC AAC TTC TCt |
| | TAC GAG GTC TTG ACC CCT TTA AAA |
| | TGG TAC CAA TCC ATC CGT CCT CCG |
| | TAT CCT AGT TAT GGC TAT GAA CCT |
| | ATG GGT GGA TGG TTG CAC CAT CAG |
| | ATT ATC CCT GTT TTA AGT CAG CAA |
| | CAC CCG CCC ACT CAT ACA TTA CAG |
| | CCT CAC CAT CAC ATT CCT GTG GTA |
| | CCT GCC CAA CAG CCG GTT ATT CCT |
| | CAA CAG CCA ATG ATG CCA GTG CCG |
| | GGT CAG CAT TCT ATG ACC CCA ATT |
| | CAA CAT CAT CAG CCT AAT TTA CCg |
| | CCA CCT GCA CAA CAG CCG TAC CAA |
| | CCC CAA CCC GTA CAA CCG CAG CCA |
| | CAT CAA CCG ATG CAG CCG CAG CCG |
| | CCA GTG CAT CCG ATG CAG CCT CTG |
| | CCA CCT CAA CCG CCA TTA CCG CCA |
| | ATG TTT CCT ATG CAA CCG CTT CCA |
| | CCG ATG TTG CCG GAT TTG ACG CTG |
| | GAA GCC TGG CCC TCT ACG GAT AAA |
| | ACC AAA CGT GAA GAG GTT GAC TAA |
| Amino acid | MVPFFPQQSGT PGMASLSLET |
| sequence of | MRQLGSLQRL NTLSQYSRYG |
| amelin-amelogenin | FGKSFNSLWM HGLLPPHSSL |
| fusion protein | PWMRPREHET QQYEYSLPVH |
| (SEQ ID NO 31) | PPPLPSQPSL KPQQPGLKPF LQSAAAT |
| | GS |
| | MPLPPHPGHPGYINFSYEVLTPLKWYQSIRP |
| | PYPSYGYEPMGGWLHHQIIPVLSQQHPPTHT |
| | LQPHHHIPVVPAQQPVIPQQPMMPVPGQHSM |
| | TPIQHHQPNLPPPAQQPYQPQPVQPQPHQPM |
| | QPQPPVHPMQPLPPQPPLPPMFPMQPLPPML |
| | PDLTLEAWPSTDKTKREEVD |

Expression of Human Amelogenin in Escherichia coli

E. coli BL21 (DE3) cells harbouring pHAP, and E. coli TG1 cells harbouring pHHAQ, were inoculated in 10 ml LB-medium (10 g/l tryptone, 10 g/l NaCl, 5 g/l yeast extract), supplemented with ampicillin (200 μg/ml), and grown overnight at 37° C. in a shaking incubator. Overnight cultures (0.5 ml) were used to inoculate 100 ml growth medium supplemented with ampicillin (200 μg/ml) in 500 ml shake flasks. Cultivation was carried out in both LB-medium and TB-medium (12 g/l tryptone, 24 g/l yeast extract, 4 ml/l glycerol, 0.017 M KH₂PO₄, 0.072 M K₂HPO₄), and amelogenin expression was induced by addition of 1 mM IPTG or by addition of lactose (2-20 mM). Induction was carried out at OD₆₀₀ between 0.2-2.0. The cells were grown at 37° C. in a shaking incubator for a total of 24 h, and samples were taken during the cultivation and analyzed on a 15% SDS-PAGE gel. Amelogenin expression was compared to control cells without the amelogenin gene.

### Purification of Recombinant Human Amelogenin

E. coli BL21 (DE3) cells harbouring pHAP were grown as described above in 100 LB-medium supplemented with ampicillin (200 μg/ml). Amelogenin expression was induced by addition of 1 mM IPTG at OD₆₀₀ of 1.5. Cells were grown for totally 24 h and was then harvested by centrifugation for 5 minutes at 5000 g. The pellet was washed by suspending it in 50 ml Buffer A (50 mM NaH₂PO₄, 300 mM NaCl, pH 7.4) and the cell suspension was divided into three new centrifuge tubes. The tubes were centrifuged 5 minutes at 5,000 g and the cell pellets were suspended in 3.3 ml of Buffer A, 0.1% HAc, or 3% HAc. The resuspended cells were sonicated for 5×20 seconds on ice and the lysates were centrifuged for 15 minutes at 15,000 g to remove cell debris and denatured proteins. The supernatants were collected and heat treated at 80° C. for 20 minutes. The samples were then centrifuged again for 15 minutes at 15,000 g to remove denatured proteins, and the supernatants were subsequently collected. During all steps samples were taken and analyzed on a 15% SDS-PAGE gel.

Another purification protocol was created to determine if the purification could be further simplified. E. coli BL21 (DE3) cells harbouring pHAP were grown in both LB-medium and TB-medium, as described above. Protein expression was induced with 1 mM IPTG at OD₆₀₀ of ˜1.9 and the cells were grown at 37° C. for a total of 24 h. The cells were then harvested by centrifugation at 5,000 g for 5 minutes and the two cell pellets were washed with 50 ml Buffer A. The cell suspensions were transferred to four centrifuge tubes, 25 ml in each tube, and centrifuged for 5 minutes at 5,000 g. The cell pellets were resuspended in 3% HAc, 5 ml for cell pellets from the LB-medium cultivation, and 10 ml for cell pellets from the TB-medium cultivation. One sample from each cultivation was sonicated for 5×20 seconds and subsequently centrifuged for 15 minutes at 15,000 g, followed by collection of the supernatant. The other two samples were kept on ice. The collected supernatants were then heat treated at 80° C. for 20 minutes, together with the untreated cells suspended in 3% HAc. All four samples were then centrifuged at 15,000 g for 15 minutes, and the supernatants were collected. During all steps samples were taken out and analyzed on a 15% SDS-PAGE gel. The same experiment was also carried out for E. coli TG1 cells harbouring pHHAQ.

The effect of different concentrations of HAc was tested by heat treatment of amelogenin expressing cells suspended in 0.1-10% HAc, followed by analysis on a 15% SDS-PAGE gel.

### Expression of Recombinant Amelin and Fusion Proteins

E. coli TG1 cells harbouring pQEam, and E. coli BL21 (DE3) cells harbouring pETam, pETam-amg, or pHAP-am, were grown overnight at 37° C. in 10 ml LB-medium supplemented with ampicillin (100 μg/ml). The overnight cultures were used to inoculate 100 ml LB-medium, supplemented with ampicillin (100 μg/ml), and the cells were grown in shake flasks at 37° C. to OD₆₀₀ of 0.6-1.0, followed by induction with 1 mM IPTG. The cells were cultured for 24 h and samples were taken for analysis on a 15% SDS-PAGE gel. Samples from fusion proteins were also analyzed by Western blotting using antibodies raised against porcine amelogenin (obtained from Biora AB, Malmö, Sweden).

### Expression and Purification of Amelogenin-Amelin Fusion Protein

E. coli BL21 (DE3) cells harbouring pHAP-am were cultivated in LB-medium (induced with 1 mM IPTG) and TB-medium (induced with 1 mM IPTG, or 5 mM lactose). The cells were grown at 37° C. for 24 hours and was then harvested, washed, resuspended in 3% HAc, heat treated and centrifuged as described above for human amelogenin. The collected supernatants were analyzed on a 15° A) SDS-PAGE gel.

### Expression of Amelogenin and Amelogenin-Fusion in Defined Medium

E. coli BL21 (DE3) cells harbouring pHAP and pHAP-am were cultivated in 100 ml defined NYAT-medium, supplemented with ampicillin (200 μg/ml). The NYAT-medium (Table 6) was composed by (per liter) 20 ml 50° A) glucose, 2 ml 1 M MgSO₄, 2 ml Trace elements, and 200 ml 5×NYAT-solution. The components were sterilized separately by autoclaving and were aseptically mixed before use. The cells were grown at 37° C. in a shaking incubator for a total of 24 h and the protein expression was induced by addition of 1 mM IPTG at OD₆₀₀ of ˜1.2.

The cells were harvested and the recombinant proteins were purified as described above by heat treatment of the cells in 3% HAc. The soluble fraction was analyzed on a 15% SDS-PAGE gel.

TABLE 6
Composition of solutions used for NYAT-medium.

| | |
|---|---|
| 5x NYAT-solution | Trace elements |

| | | | |
|---|---|---|---|
| (NH₄)₂SO₄ | 10 g/l | CaCl₂•2 H₂O | 0.5 g/l |
| K₂HPO₄ | 73 g/l | FeCl₃•6 H₂O | 16.7 g/l |
| NaH₂PO₄•H₂O | 16 g/l | ZnSO₄•7 H₂O | 0.18 g/l |
| (NH₄)₂•H•citrate | 2.5 g/l | CuSO₄•5 H₂O | 0.16 g/l |
| | | MnSO₄•H₂O | 0.15 g/l |
| | | CoCl₂•6 H₂O | 0.18 g/l |
| | | Na₂EDTA | 20.1 g/l |

### Results

### Expression of Recombinant Human Amelogenin

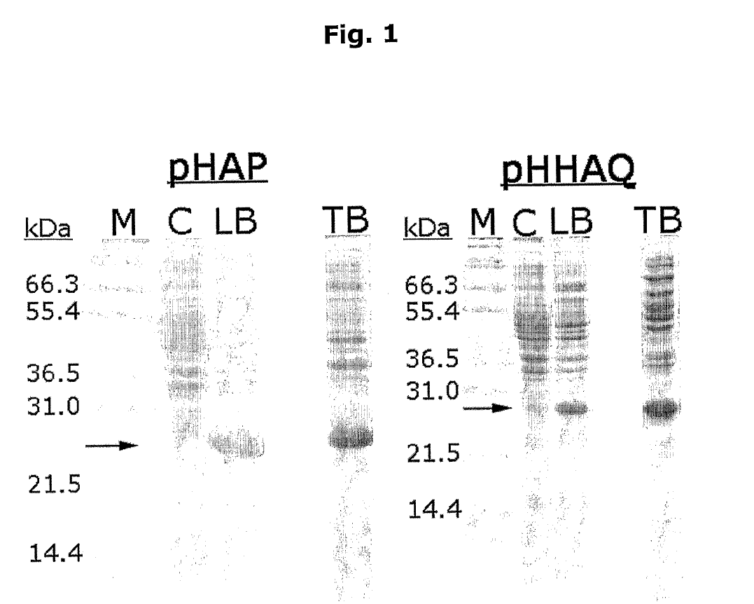

A gene encoding the 175 aa human amelogenin was synthesized and the expression vectors pHAP and pHHAQ were created. The vector pHAP generates an untagged amelogenin, and pHHAQ generates an amelogenin with the N-terminal tag MRGSHHHHHHGS. E. coli cells harbouring pHAP or pHHAQ expresses amelogenin when cultivated in both LB-medium and TB-medium (FIG. 1). Expression could be induced by addition of IPTG or lactose.

### Purification of Recombinant Human Amelogenin

Recombinant human amelogenin was expressed in E. coli BL21 (DE3) cells from expression vector pHAP. The cells were disrupted by sonication in three different solutions (Buffer A, 0.1% HAc or 3% HAc) and subsequently centrifuged. The lysates were then heat treated at 80° C. and centrifuged, and the supernatants were collected.

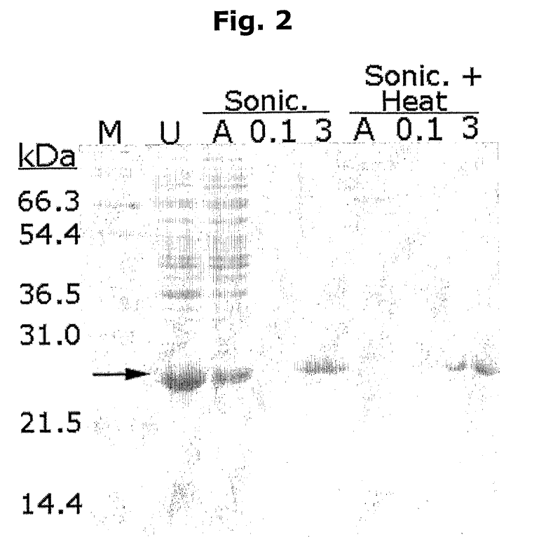

As seen in FIG. 2, sonication in Buffer A generates a protein extract containing practically all proteins inside the cells, whereas almost no proteins are soluble after sonication in 0.1% HAc. Sonication in 3% HAc shows one band for amelogenin in the soluble fraction. Heating of the Buffer A-extract denatures a large fraction of the proteins, there among amelogenin. The amount of soluble protein left after heating of the 0.1% HAc-extract is practically undetectable on the SDS-PAGE gel. The amelogenin in the 3% HAc-extract is basically not affected by the heating and remains in solution.

An alternative protocol for amelogenin purification was created in order to determine if the purification could be further simplified. Amelogenin expressing cells, E. coli BL21 (DE3) harbouring pHAP, were subjected to sonication in 3% HAc, centrifugation, followed by heat treatment of the lysate at 80° C. This sample was then compared to a sample where untreated amelogenin expressing cells were subjected to the heat treatment in 3% HAc, without prior disruption. Amelogenin purity was then analysed by SDS-PAGE.

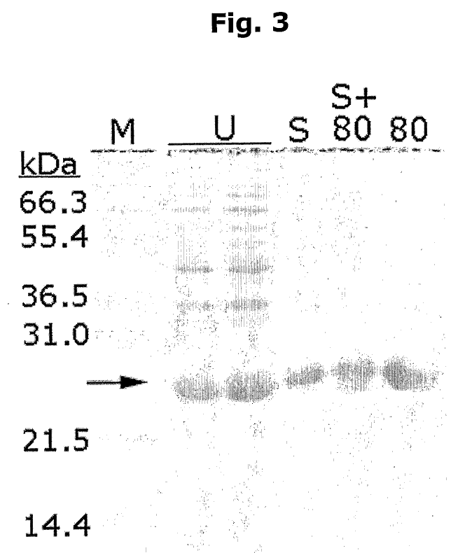

As seen in FIG. 3, sonication in 3% HAc denatures many of the contaminating proteins. Subsequent heat treatment of the sonicated sample does not seem to give a further separation of contaminating proteins. Heat treatment of untreated cells in 3% HAc gives the most pure amelogenin, and very few contaminating proteins are visible on the gel.

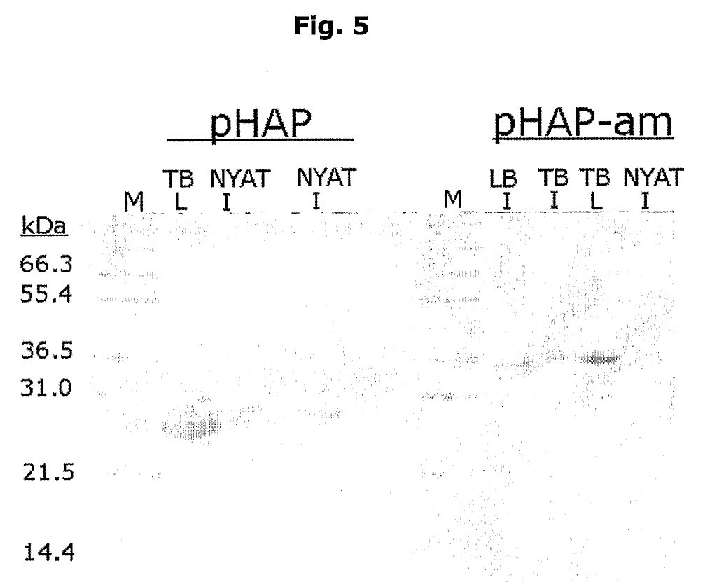

The same results were obtained both when LB-medium and TB-medium was used for cell culture, and the results were also the same when using cells harbouring pHHAQ. Also NYAT-medium could be used to express amelogenin for purification (FIG. 5).

Heat treatment of amelogenin expressing cells in different concentrations of HAc showed that even 0.1 HAc was enough to denature practically all host cell proteins. However no amelogenin could be detected in the soluble fraction until the concentration of HAc was increased to 0.25-0.5%. Concentrations between 1-5% seemed to give the highest amount of amelogenin in the soluble fraction.

### Expression of Recombinant Amelin and Fusion Proteins

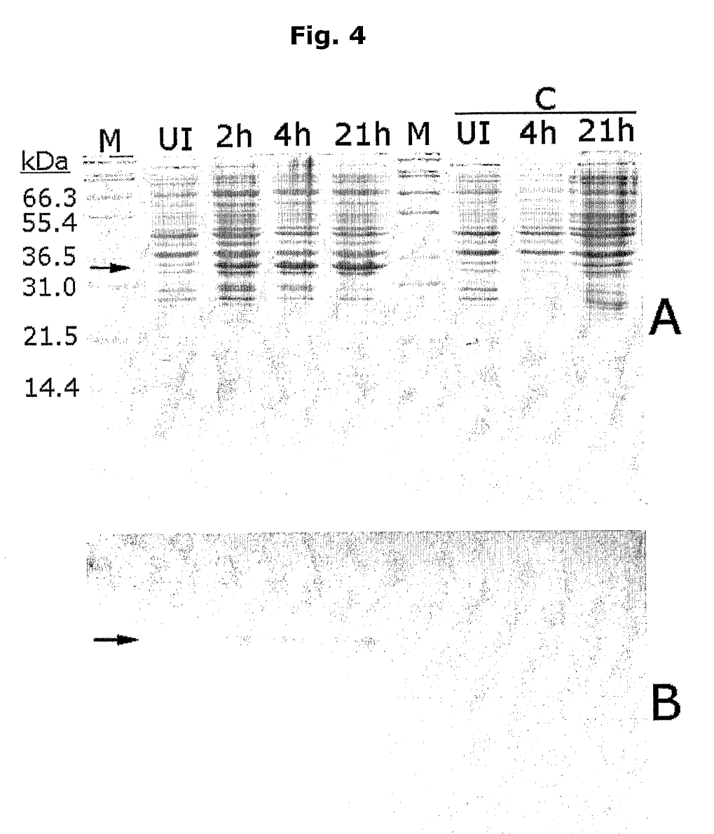

A synthetic gene encoding the first 107 amino acids in human amelin (without the signal peptide) was created and used to construct the expression vectors pQEam and pETam. The plasmid pETam generates an untagged protein, and pQEam generates a protein with the N-terminal tag MRGSHHHHHHGS. E. coli cells harbouring the two expression vectors were cultivated and analysed by SDS-PAGE, but no amelin expression could by detected. The synthetic genes for amelogenin and amelin were used to create two fusion protein expression vectors: pETam-amg, expressing an amelin-amelogenin fusion, and pHAP-am, expressing an amelogenin-amelin fusion. Expression of the amelin-amelogenin fusion protein from the vector pETam-amg did not generate any detectable recombinant protein. The expression of the amelogenin-amelin fusion however, resulted in a fusion protein detectable by analysis by SDS-PAGE and Western blot (FIG. 4).

Expression could be induced with IPTG or lactose, and the cultivation could be carried out in either LB-medium, TB-medium or NYAT-medium (FIG. 5).

### Purification of Amelogenin-Amelin Fusion Protein

Cells expressing the amelogenin-amelin fusion were cultivated and harvested, and the fusion protein was purified in the same manner as for amelogenin by heat treatment of the cells in 3% HAc. SDS-PAGE analysis (FIG. 5) shows that the fusion protein is present in the soluble fraction after the heat treatment, and that practically all host cell proteins have been removed.

### DISCUSSION & CONCLUSIONS

Synthetic genes encoding the human 175 amino acid amelogenin (without signal peptide), and the 107 N-terminal amino acids of human amelin (without signal peptide) were constructed and expressed in E. coli. Fusion proteins of between amelogenin and amelin were also created and expressed in E. coli. Amelogenin and the amelogenin-amelin fusion protein were produced by the cells, but production of amelin and the amelin-amelogenin fusion protein could not be detected. Production of amelogenin and amelogenin-amelin fusion could be carried out in both complex growth mediums (LB and TB) and in defined medium (NYAT), and protein expression could be induced by IPTG or lactose.

The problem of expressing amelin and amelin-amelogenin fusion indicates that amelin is a tricky protein to produce in E. coli, despite the fact that the gene was codon optimized. The reason for this could be due to a very low stability or unfavourable conformation of the mRNA, or that the protein in itself is unstable inside the E. coli cell and thereby rapidly degraded. Amelogenin on the other hand is produced at a high level (FIG. 1), both with and without the tag, suggesting that this protein is relatively stable inside the cells. The amelogenin-amelin fusion is also produced by the cells (FIG. 4), but not to as high levels as for amelogenin alone. Fusion to amelogenin seems to help the expression of amelin, maybe by facilitate transcription or translation, or by stabilize the protein once expressed. It is fully possible that other proteins than amelin could be expressed more easily by fusing it to amelogenin.

Production of a pure recombinant protein is a multi step process including cell cultivation and harvesting, cell disruption, and various steps of protein purification. The purification process generally starts with a crude extract containing the protein of interest (POI) together with a lot of contaminants, mainly host cell proteins. The crude extract is obtained by disruption of the cells using mechanical, chemical or enzymatic methods, followed by separation of the insoluble fraction. Separation of the POI from the host cell proteins in the crude extract is a multi step process that starts with a couple of primary steps, often chromatography based, in order to remove the bulk of contaminating proteins. This is followed by a number of polishing steps, to further remove unwanted proteins and other contaminants, in order to obtain the POI with sufficient purity.

Purification of amelogenin from E. coli cells started by disrupting the cells by sonication in different solutions, in order to create a crude extract. It was found that sonication in 3% HAc precipitated a lot of the host cell proteins (FIG. 2), most certainly because of the low pH. The amelogenin however, was still left in solution and could easily be isolated by removing the insoluble fraction. Sonication in Na-phosphate buffer (Buffer A) left practically all proteins soluble, suggesting that this buffer is a poorer choice for amelogenin purification. Subsequent heat treatment at 80° C. of the crude extracts showed that amelogenin dissolved in 3% HAc was resistant to the high temperature, but when Na-phosphate buffer was used this temperature resistance was not observed and amelogenin precipitated together with many of the host cell proteins. This indicates that the temperature resistance of amelogenin is pH dependant. In order to see if the isolation of recombinant amelogenin could be made even easier, untreated cells were heated to 80° C. in 3% HAc, without prior disruption. The aim was to make the cells disrupt by the heat treatment, and make the host cell proteins precipitate by the low pH and the heat, leaving only amelogenin left in solution. This procedure would combine cell disruption with purification of recombinant amelogenin. FIG. 3 shows the results from using this approach compared to using a separate step for cell disruption, and demonstrates that the combined lysis/purification step generates a more pure amelogenin. A separate cell lysis step, followed by heat treatment of the crude extract would also be more laborious than direct heat treatment of the harvested cells. The purity of the crude extract obtained from sonication of the cells in 3 HAc is not significantly improved by the subsequent heat treatment, suggesting that it is the low pH more than the high temperature that denatures the host cell proteins. A heating step could nevertheless be desirable in a purification process since it could help degrade other contaminants and provide sterilization of the product.

The method to heat treat cells in an acidic solution could most likely be used also for other recombinantly expressed proteins. The protein must be soluble, but not necessarily active, in the solution used and at the high temperature. The exact temperature and solution used may have to be modified for the specific protein. This method generates a POI almost free from host cell proteins in only one step, starting with untreated cells, and should be very simple to perform also in a large scale. The total purification process should be shortened since cell disruption and primary purification could be made to one unit.

Purification of the amelogenin-amelin fusion protein was carried out in same manner as for amelogenin by heat treatment of the cells at 80° C. in 3% HAc. This procedure showed successful also for the fusion protein (FIG. 5), and a very pure protein was obtained from the cells by only one step. A low amount of contaminants are visible, probably due to degradation of the amelin part of the fusion. It is fully possible that purification of other proteins, using heat treatment at low pH, could be promoted by fusing the POI to amelogenin, even if the POI alone not withstands this treatment. Thus, amelogenin seems like an attractive fusion partner, both to facilitate expression and purification.

Purification of recombinant human amelogenin and amelogenin-amelin fusion protein could be carried out by a simple method, where the protein expressing cells were subjected to heat in a low pH solution. This one step procedure generated proteins practically free from contaminating host cell proteins. The simplicity, ease and speed of this method should make it an attractive alternative in production of recombinant proteins.

### LIST OF REFERENCES

1. Middelberg A., “Process-scale disruption of microorganisms”, Biotechnology Advances, 1995, Vol 13, No. 3, pp. 491-551
2. De Marco, A. et al., “Recombinant proteins fused to thermostable partners can be purified by heat incubation.” Journal of Biotechnology 107, (2004) 125-133.
3. Hammarström et al., 1997, Journal of Clinical Periodontology 24, 658-668).
4. Lyngstadaas et al., 2001, Journal of Clinical Periodontology 28, 181-188).
5. Fincham et al, 1993,
6. Brennan, Chemical and Engineering News, Oct. 14, 1996.
7. Kojima et al, “Midkine is a heat and acid stable polypeptide capable of enhancing plasminogen activator activity and neurite outgrowth extension” Biochemical and Biophysical Research Communications, Vol. 216, nr 2, p. 574-581, 1995).
8. Huang et al, “A highly chemostable and thermostable endo-β-glucanase from the thermoacidophilic archaeon Sulfulobus solfataricus”, Biochem. J. (2005), 385, 581-588).
9. Kaneko et al, “Characterization of Chemostable Glucose Isomerase from Streptomyces sp., and Development of Single-Step Processes for High-Fructose Corn Sweetener (HFCS) production” Biosci. Biotechnol. Biochem., 64 (5), 940-947 (2005).
10. Ten Cate: Oral Histology, 1994.
11. Robinson: Eur. J. Oral Science, January 1998, 106 Suppl. 1:282-91.
12. Gestrelius S, Lyngstadaas S P, Hammarstrøm L. Emdogain—periodontal regeneration based on biomimicry. Clin Oral Invest 4:120-125 (2000).
13. Dayhoff, Schwartz, and Orcutt (1978) Atlas Protein Seq. Struc. 5:345-352,
14. Henikoff and Henikoff (1992) Proc Natl Acad Sci USA 89(22):10915-9.
15. Simon R J et al., PNAS (1992) 89(20), 9367-9371.
16. Norwell D C, Trends Biotechnol. (1995) 13(4), 132-134.
17. J. Amer. Dent. Ass. 32: 397-418, 1945.
18. Sundell S, Hereditary amelogenesis imperfecta. An epidemiological, genetic and clinical study in a Swedish child population, Swed Dent J Suppl 1986; 31: 1-38.

1. A process for purifying a recombinant protein, comprising
a) suspending host cells expressing said protein in acetic acid and incubating said suspension at a temperature of between 60-100° C.,
b) separating, and
c) collecting the soluble fraction of the suspension, containing the recombinant protein.

2. A process for purifying a recombinant protein, comprising
a) harvesting a cell culture of host cells expressing said protein,
b) resuspending said host cells in acetic acid and incubating said suspension at 60-100° C.,
c) separating, and
d) collecting the soluble fraction of the suspension, containing the recombinant protein.

3. (canceled)

4. (canceled)

5. (canceled)

6. A process for purifying a recombinant protein according to claim 1, wherein the soluble fraction of the suspension, containing the recombinant protein, is subsequently subjected to one or more additional purification step(s).

7. A process for purifying a recombinant protein according to claim 1, wherein the host cells are resuspended in 0.15-5% acetic acid.

8. (canceled)

9. (canceled)

10. A process for purifying a recombinant protein according to claim 1, wherein the suspension displays a pH below or equal to 5.

11. A process for purifying a recombinant protein according to claim 1, wherein the suspension displays a pH below or equal to 3.

12. A process for purifying a recombinant protein according to claim 1, wherein the incubation is performed at 75-85° C.

13. (canceled)

14. (canceled)

15. (canceled)

16. A process for purifying a recombinant protein according to claim 1, wherein the host cells are prokaryotic cells or eukaryotic cells.

17. A process for purifying a recombinant protein according to claim 1, wherein the host cells are selected from the group consisting of bacterial cells, insect cells, yeast, mammal cells, and plant cells.

18. A process for purifying a recombinant protein according to any claim 1, wherein the host cells are Escherichia coli cells.

19. (canceled)

20. (canceled)

21. A process for purifying a recombinant protein according to claim 1, wherein the expression of said recombinant protein in said host cells, is induced by addition of lactose to the medium.

22. A process for purifying a recombinant protein according to claim 1, wherein the expression of said recombinant protein in said host cells, is induced by addition of IPTG to the medium.

23. A process for purifying a recombinant protein according to claim 1, wherein the host cells are transformed and/or transfected with a vector for expression of said recombinant protein.

24. (canceled)

25. A process for purifying a recombinant protein according to claim 1, wherein the recombinant protein is selected from the group consisting of thermostable proteins, acid stable proteins and proteins that are soluble at a low pH and/or at an elevated temperature.

26. A process for purifying a recombinant protein according to claim 1, wherein the protein is a fusion protein comprising at least one fragment of a protein selected from the group consisting of thermostable proteins, acid stable proteins and proteins that are soluble at a low pH and/or at an elevated temperature.

27. A process for purifying a recombinant protein according to claim 1, wherein the protein is a fusion protein comprising at least one fragment of a thermostable protein.

28. A process for purifying a recombinant protein according to claim 1, wherein the protein is selected from the group consisting of hormones, parathyroid hormones, growth hormones, gonadotropins, insulin, ACTH, prolactin, placental lactogen, melanocyte stimulating hormone, thyrotropin, calcitonin, enkephalin, angiotensin, cytokines human serum albumin, bovine serum albumin, ovalbumin, glucose isomerase, α-amylase, and endo-β-glucanase, growth hormone (GH), IGF-1, IGF-2, PTH, PGE₂, TGF-beta, TGF-alpha, bEGF, EGF, PDGF-AB, PDGF-BB, osteoprotegerin (OPG), osteopontin (OP), FGF-1, FGF-2, thyroid hormone, BMP-2, BMP-3, BMP-4, BMP-6, BMP-7, VEGF, 1.25(OH)₂ vitamin D₃, Caclitonin, IFN-gamma, OCN (osteocalcin), ON (osteonectin), OP-1 (osteogenic protein-1), NGF, collagen, fibronectin, fibrinogen, thrombin, factor XIII.

29. A process for purifying a recombinant protein according to claim 1, wherein the protein is selected from the group consisting of an enamel matrix, enamel matrix derivative and/or an enamel matrix protein.

30. A process for purifying a recombinant protein according to claim 1, wherein the protein is selected from the group consisting of amelogenin, proline-rich non-amelogenins, amelin, tuftelin, tuft proteins, serum proteins, salivary proteins, ameloblastin, sheathlin, and derivatives thereof, and mixtures thereof.

31. A process for purifying a recombinant protein according to claim 1, wherein the recombinant protein is amelogenin.

32. A process for purifying a recombinant protein according to claim 1, wherein the protein is a fusion protein comprising at least one fragment of amelogenin.

33. A process for purifying a recombinant protein according to claim 1, wherein the protein is a fusion protein comprising at least a fragment of amelogenin and a fragment of a protein selected from the group consisting of proline-rich non-amelogenins, amelin, tuftelin, tuft proteins, serum proteins, salivary proteins, ameloblastin, sheathlin, and derivatives thereof, and mixtures thereof.

34. A process for purifying a recombinant protein according to claim 1, wherein the protein is a fusion protein comprising at least one fragment of amelin.

35. A process for purifying a recombinant protein according to claim 1, wherein the protein is a fusion protein comprising at least one fragment of amelogenin and at least one fragment of amelin.

36. A process for purifying a recombinant protein according to claim 1, wherein;
the protein is a protein encoded by SEQ ID NO 1, or a fragment thereof;
the protein is a protein encoded by SEQ ID NO 2, or a fragment thereof;
the protein is a protein encoded by SEQ ID NO 3, or a fragment thereof; or
the protein is a fusion protein comprising the amino acid sequence as shown in SEQ ID NO 4.

37. (canceled)

38. (canceled)

39. (canceled)

40. A vector for expressing a recombinant protein wherein said vector is pHHAQ, pETam, pQEam or pHAP.

41. A vector for expressing a recombinant protein wherein said vector is pHAP-am or pETam-amg.

42. Use of a vector according to claim 40-41 for expressing a recombinant protein in a host cell.

43. A host cell transfected and/or transformed with a vector according to claim 40-41.

44. A purified recombinant protein expressed by a host cell according to claim 43.

45. A recombinant protein according to claim 44 for use as a medicament.

46. A recombinant protein, purified by a process described in any of claims 28-39.

47. A recombinant protein, purified by a process described in any of claims 28-39 for use as a medicament.
